(19) United States
(12) Patent Application Publication
(10) Pub. No.: US 2025/0222032 A1
(43) Pub. Date: Jul. 10, 2025

(54) T CELL RECEPTORS TARGETING RAS MUTATIONS AND USES THEREOF

(71) Applicants: Memorial Sloan-Kettering Cancer Center, New York, NY (US); Sloan-Kettering Institute for Cancer Research, New York, NY (US); Memorial Hospital for Cancer and Allied Diseases, New York, NY (US)

(72) Inventors: Christopher A. Klebanoff, New York, NY (US); Smita S. Chandran, Long Island City, NY (US)

(73) Assignees: Memorial Sloan-Kettering Cancer Center, New York, NY (US); Sloan-Kettering Institute for Cancer Research, New York, NY (US); Memorial Hospital for Cancer and Allied Diseases, New York, NY (US)

(21) Appl. No.: 19/056,365

(22) Filed: Feb. 18, 2025

Related U.S. Application Data
(63) Continuation of application No. PCT/US2023/030031, filed on Aug. 11, 2023.
(60) Provisional application No. 63/399,410, filed on Aug. 19, 2022.

(51) Int. Cl. A61K 35/17; A61K 40/11; A61K 40/15; A61K 40/32; A61K 40/42; C07K 14/705; C07K 14/73; C07K 16/40; C12N 5/0783; C12N 15/86

(52) U.S. Cl. CPC A61K 35/17; A61K 40/11; A61K 40/15; A61K 40/32; A61K 40/421; A61K 40/4253; C07K 14/70514; C07K 14/70517; C07K 16/40; C12N 5/0636; C12N 5/0646; C12N 15/86; A61K 2239/28; C07K 2317/565; C12N 2740/13043

(57) ABSTRACT

The presently disclosed subject matter provides novel T cell receptors (TCRs) that target a mutated RAS protooncogene. The presently disclosed subject matter further provides cells comprising such TCRs, and methods of using such cells for treating cancers associated with RAS.

### CROSS-REFERENCE TO RELATED APPLICATIONS

This application is a continuation application of International Patent Application No. PCT/US2023/030031, filed Aug. 11, 2023, which claims priority to U.S. Provisional Application No. 63/399,410, filed Aug. 19, 2022, the contents of each of which are incorporated by reference in their entireties, and to each of which priority is claimed.

### SEQUENCE LISTING

A Sequence Listing conforming to the rules of WIPO Standard ST.26 is hereby incorporated by reference. Said Sequence Listing has been filed as an electronic document via PatentCenter encoded as XML in UTF-8 text. The electronic document, created on Feb. 17, 2025, is entitled “0727341738”, and is 35,016 bytes in size.

### 1. INTRODUCTION

The presently disclosed subject matter provides novel T cell receptors (TCRs) that target mutated RAS proto-oncogenes. The presently disclosed subject matter further provides cells comprising such TCRs, and methods of using such cells for treating cancers associated with mutated RAS.

### 2. BACKGROUND OF THE INVENTION

Cell-based immunotherapy is a therapy with curative potential for treating cancers. Immunoresponsive cells (e.g., T cells) may be modified to target tumor antigens through the introduction of genetic material coding for TCRs specific to selected antigens. Targeted T cell therapy using specific TCRs has shown clinical success in treating diverse solid and hematologic malignancies.

Collectively, the RAS proteins are the most mutated family of oncoproteins in human cancer. Patients with oncogenic mutations encoding a RAS protein (e.g., KRAS, NRAS, and HRAS) typically respond poorly to standard therapies. Activating oncogenic RAS mutations are frequently observed at residue positions 12, 13 and 61 in cancer patients. Among these, G12 is the most frequently mutated residue (89%) and it most often mutates to aspartate (G12D), valine (G12V), or cysteine (G12C). Accordingly, there are needs for novel therapeutic strategies to identify TCRs targeting epitopes derived from mutated RAS proteins. Further, there is unmet need for developing strategies capable of inducing potent cancer eradication with minimal toxicity and immunogenicity.

### 3. SUMMARY OF THE INVENTION

The presently disclosed subject matter provides T cell receptors (TCRs) targeting a RAS peptide that comprises a mutation. In certain embodiments, the RAS peptide comprises a G12 mutation. In certain embodiments, the RAS peptide comprises a G12D mutation. In certain embodiments, the RAS peptide is a 9-mer or a 10-mer. In certain embodiments, the RAS peptide is a 10-mer. In certain embodiments, the RAS peptide comprises or consists of the amino acid sequence set forth in SEQ ID NO: 1 or SEQ ID NO: 2. In certain embodiments, the RAS peptide comprises or consists of the amino acid sequence set forth in SEQ ID NO: 2.

In certain embodiments, the RAS peptide comprises a G12V mutation. In certain embodiments, the RAS peptide is a 9-mer or a 10-mer. In certain embodiments, the RAS peptide is a 10-mer. In certain embodiments, the RAS peptide comprises or consists of the amino acid sequence set forth in SEQ ID NO: 4 or SEQ ID NO: 5.

In certain embodiments, the RAS peptide comprises a G12C mutation. In certain embodiments, the RAS peptide is a 10-mer. In certain embodiments, the RAS peptide comprises or consists of the amino acid sequence set forth in SEQ ID NO: 6.

In certain embodiments, the RAS peptide is associated with an HLA class I complex. In certain embodiments, the HLA class I complex is selected from an HLA-A, an HLA-B, and an HLA-C. In certain embodiments, the HLA class I complex is an HLA-A. In certain embodiments, the HLA-A is an HLA-A*03 superfamily member. In certain embodiments, the HLA-A*03 superfamily is selected from the group consisting of HLA-A*03, HLA-A*11, HLA-A*31, HLA-A*33, HLA-A*66, HLA-A*68 and HLA-A*74. In certain embodiments, the HLA-A*03 superfamily member is HLA-A*11.

In certain embodiments, the TCR comprises an extracellular domain that binds to the RAS peptide, wherein the extracellular domain comprises an α chain and a β chain, wherein the α chain comprises an α chain variable region and α chain constant region, and the β chain comprises a β chain variable region and a β chain constant region.

In certain embodiments, the α chain variable region comprises a CDR3 comprising the amino acid sequence set forth in SEQ ID NO: 18 or a conservative modification thereof, and the β chain variable region comprises a CDR3 comprising the amino acid sequence set forth in SEQ ID NO: 21 or a conservative modification thereof.

In certain embodiments, the α chain variable region comprises a CDR2 comprising the amino acid sequence set forth in SEQ ID NO: 17 or a conservative modification thereof, and the β chain variable region comprises a CDR2 comprising the amino acid sequence set forth in SEQ ID NO: 20 or a conservative modification thereof.

In certain embodiments, the α chain variable region comprises a CDR1 comprising the amino acid sequence set forth in SEQ ID NO: 16 or a conservative modification thereof, and the β chain variable region comprises a CDR1 comprising the amino acid sequence set forth in SEQ ID NO: 19 or a conservative modification thereof.

In certain embodiments, the α chain variable region comprises a CDR1 comprising the amino acid sequence set forth in SEQ ID NO: 16 or a conservative modification thereof, a CDR2 comprising the amino acid sequence set forth in SEQ ID NO: 17 or a conservative modification thereof, and a CDR3 comprising the amino acid sequence set forth in SEQ ID NO: 18 or a conservative modification thereof; and the β chain variable region comprises a CDR1 comprising the amino acid sequence set forth in SEQ ID NO: 19 or a conservative modification thereof, a CDR2 comprising the amino acid sequence set forth in SEQ ID NO: 20 or a conservative modification thereof, and a CDR3 comprising the amino acid sequence set forth in SEQ ID NO: 21 or a conservative modification thereof.

In certain embodiments, the α chain variable region comprises a CDR1 comprising the amino acid sequence set forth in SEQ ID NO: 16, a CDR2 comprising the amino acid sequence set forth in SEQ ID NO: 17, and a CDR3 comprising the amino acid sequence set forth in SEQ ID NO: 18; and the β chain variable region comprises a CDR1 comprising the amino acid sequence set forth in SEQ ID NO: 19, a CDR2 comprising the amino acid sequence set forth in SEQ ID NO: 20, and a CDR3 comprising the amino acid sequence set forth in SEQ ID NO: 21.

In certain embodiments, the α chain variable region comprises an amino acid sequence that is at least about 80% homologous or identical to the amino acid sequence set forth in SEQ ID NO: 7. In certain embodiments, the α chain variable region comprises the amino acid sequence set forth in SEQ ID NO: 7. In certain embodiments, the β chain variable region comprises an amino acid sequence that is at least about 80% homologous or identical to the amino acid sequence set forth in SEQ ID NO: 8. In certain embodiments, the β chain variable region comprises the amino acid sequence set forth in SEQ ID NO: 8. In certain embodiments, the α chain variable region comprises an amino acid sequence that is at least about 80% homologous or identical to the amino acid sequence set forth in SEQ ID NO: 7, and the β chain variable region comprises an amino acid sequence that is at least about 80% homologous or identical to the amino acid sequence set forth in SEQ ID NO: 8. In certain embodiments, the α chain variable region comprises the amino acid sequence set forth in SEQ ID NO: 7, and the β chain variable region comprises the amino acid sequence set forth in SEQ ID NO: 8.

In certain embodiments, the TCR is recombinantly expressed, and/or expressed from a vector.

In certain embodiments, the α chain constant region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 9, SEQ ID NO: 10, or SEQ ID NO: 25. In certain embodiments, the α chain constant region comprises the amino acid sequence set forth in SEQ ID NO: 9, SEQ ID NO: 10, or SEQ ID NO: 25. In certain embodiments, the β chain constant region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 11, SEQ ID NO: 12, SEQ ID NO: 13, or SEQ ID NO: 14. In certain embodiments, the β chain constant region comprises the amino acid sequence set forth in SEQ ID NO: 11, SEQ ID NO: 12, SEQ ID NO: 13, or SEQ ID NO: 14. In certain embodiments, the TCR is recombinant.

The presently disclosed subject matter also provides a T cell receptor (TCR) that comprises an extracellular domain that binds to the same RAS peptide as a reference TCR or a functional fragment thereof, wherein the reference TCR or functional fragment thereof comprises an α chain variable region and a β chain variable region, wherein the α chain variable region comprises a CDR1 comprising the amino acid sequence set forth in SEQ ID NO: 16, a CDR2 comprising the amino acid sequence set forth in SEQ ID NO: 17, and a CDR3 comprising the amino acid sequence set forth in SEQ ID NO: 18; and the β chain variable region comprises a CDR1 comprising the amino acid sequence set forth in SEQ ID NO: 19, a CDR2 comprising the amino acid sequence set forth in SEQ ID NO: 20, and a CDR3 comprising the amino acid sequence set forth in SEQ ID NO: 21.

The presently disclosed subject matter provides nucleic acids encoding the TCRs disclosed herein. The presently disclosed subject matter further provides cells comprising the TCR disclosed herein or the nucleic acids disclosed herein. In certain embodiments, the cell is transduced with the TCR. In certain embodiments, the TCR is constitutively expressed on the surface of the cell. In certain embodiments, the cell is an immunoresponsive cell. In certain embodiments, the cell is selected from the group consisting of a T cell, a Natural Killer (NK) cell, and a pluripotent stem cell from which a lymphoid cell may be differentiated. In certain embodiments, the cell is a T cell. In certain embodiments, the T cell is selected from the group consisting of a cytotoxic T lymphocyte (CTL), a regulatory T cell, a γδ T cell, a Natural Killer-T cell (NK-T), a stem cell memory T cell (T_{SCM}), a central memory T cell (T_{CM}), and an effector memory T cell (T_{EM}). In certain embodiments, the T cell is a γδ T cell. In certain embodiments, the T cell is a NK-T cell. In certain embodiments, the T cell is an NK cell.

In certain embodiments, the TCR or nucleic acid is integrated at a locus within the genome of the cell (e.g., T cell). In certain embodiments, the locus is selected from a TRAC locus, a TRBC locus, a TRDC locus, and a TRGC locus. In certain embodiments, the locus is a TRAC locus or a TRBC locus. In certain embodiments, the locus is selected from PDCD1 locus, a CBLB locus, a CISH locus, and a RASA2 locus. In certain embodiments, the locus is selected from PDCD1 locus. In certain embodiments, the locus is selected from CBLB locus. In certain embodiments, the locus is selected from CISH locus. In certain embodiments, the locus is selected from RASA2. In certain embodiments, the locus is a genomic safe harbor.

In certain embodiments, the cell further comprises at least one recombinant or exogenous coreceptor. In certain embodiments, the co-receptor is a CD8 co-receptor. In certain embodiments, the co-receptor is a CD4 co-receptor.

The presently disclosed subject matter also provides compositions comprising the cells disclosed herein. In certain embodiments, the composition is a pharmaceutical composition further comprising a pharmaceutically acceptable carrier.

Furthermore, the presently disclosed subject matter provides vectors comprising the nucleic acids disclosed herein. In certain embodiments, the vector is a 7-retroviral vector.

Additionally, the presently disclosed subject matter provides methods for producing a cell that binds to a RAS peptide that comprises a G12 mutation. In certain embodiments, the method comprises introducing into the cell the nucleic acid or the vector disclosed herein.

Furthermore, the presently disclosed subject matter provides methods of treating and/or preventing a tumor associated with RAS in a subject. In certain embodiments, the method comprises administering to the subject the cells or the compositions disclosed herein. In certain embodiments, the tumor is associated with a RAS mutation. In certain embodiments, the RAS mutation is a G12D mutation. In certain embodiments, the RAS mutation is a G12V mutation. In certain embodiments, the RAS mutation is a G12C mutation.

In certain embodiments, the tumor is selected from the group consisting of pancreatic cancer, breast cancer, endometrial cancer, cervical cancer, anal cancer, bladder cancer, colorectal cancer, cholangiocarcinoma/bile duct cancer, lung cancer, ovarian cancer, esophageal cancer, gastric cancer, head and neck squamous cell carcinoma, non-melanoma skin cancer, salivary gland cancer, melanoma, and multiple myeloma. In certain embodiments, the tumor is pancreatic cancer.

In certain embodiments, the tumor is colorectal cancer. In certain embodiments, the subject is a human. In certain embodiments, the subject comprises an HLA-A. In certain embodiments, the HLA-A is an HLA-A*03 superfamily member. In certain embodiments, the HLA-A*03 superfamily member is selected from the group consisting of HLA-A*03, HLA-A*11, HLA-A*31, HLA-A*33, HLA-A*66, HLA-A*68 and HLA-A*74. In certain embodiments, the HLA-A*03 superfamily member is HLA-A*11.

Furthermore, the presently disclosed subject matter provides uses of the cells or compositions disclosed herein for treating and/or preventing a tumor associated with RAS in a subject. In certain embodiments, the tumor is associated with a RAS mutation. In certain embodiments, the RAS mutation is a G12D mutation. In certain embodiments, the RAS mutation is a G12V mutation. In certain embodiments, the RAS mutation is a G12C mutation. In certain embodiments, the tumor is selected from the group consisting of pancreatic cancer, breast cancer, endometrial cancer, cervical cancer, anal cancer, bladder cancer, colorectal cancer, cholangiocarcinoma/bile duct cancer, lung cancer, ovarian cancer, esophageal cancer, gastric cancer, head and neck squamous cell carcinoma, nonmelanoma skin cancer, salivary gland cancer, melanoma, and multiple myeloma. In certain embodiments, the tumor is colorectal cancer. In certain embodiments, the subject is a human. In certain embodiments, the subject comprises an HLA-A. In certain embodiments, the HLA-A is an HLA-A*03 superfamily member. In certain embodiments, the HLA-A*03 superfamily member is selected from the group consisting of HLA-A*03, HLA-A*11, HLA-A*31, HLA-A*33, HLA-A*66, HLA-A*68 and HLA-A*74. In certain embodiments, the HLA-A*03 superfamily member is HLA-A*11.

### 4. BRIEF DESCRIPTION OF THE FIGURES

The following Detailed Description, given by way of example but not intended to limit the invention to specific embodiments described, may be understood in conjunction with the accompanying drawings.

FIGS. 1A-1C illustrate that RAS is the most frequently mutated oncogene family in cancer. FIG. 1A shows a graph of the most frequest somatic mutations observed in KRAS polypeptides as reported by The Cancer Genome Atlas (TCGA). Missense mutations, nonsense mutations, and frameshift mutations are indicated. FIG. 1B shows a graphical comparison of the amino acid sequence homology and location of hotspot mutations in the RAS family of oncoproteins. *=location of hotspot mutations; vertical bar=site of sequence variation between RAS family members. Zoom area shows the sequence of the hypervariable region of all four RAS proteins. FIG. 1C shows charts indicating the frequencies of RAS mutations in different cancers.

FIGS. 2A and 2B illustrate detection and measurement of co-receptor dependency of a patient-derived TCR that recognizes a RAS public neoantigen. FIG. 2A shows dual dextramer labeling of T cells. FIG. 2B shows specificity of TCR T3 transduced T cells.

FIGS. 3A and 3B illustrate cross-protection of RAS public neoantigen-specific TCR T3 against alternative mutant RAS variants. FIG. 3A shows bar graphs demonstrating recognition of the RAS isoform family by TCR T3. FIG. 3B shows bar graphs demonstrating recognition of the alternative p.G12 substitution by TCR T3.

FIG. 4 illustrates peptide-titration of T cells transduced with TCR T3.

### 5. DETAILED DESCRIPTION OF THE INVENTION

The presently disclosed subject matter provides TCRs, targeting RAS comprising a mutation, e.g., a G12D mutation. Furthermore, the presently disclosed subject matter provides cells (e.g., T cells) comprising the RAS-targeted TCRs, and methods of using such cells for treating tumors associated with RAS mutation(s).

For purposes of clarity of disclosure and not by way of limitation, the detailed description is divided into the following subsections:
5.1. Definitions;
5.2. RAS;
5.3. TCRs;
5.4. Cells;
5.5. Nucleic Acids and Genetic Modifications of Cell;
5.6. Formulations and Administration;
5.7. Methods of Treatments;
5.8. Diagnostic and Prognostic Methods;
5.9 Kits; and
5.10. Exemplary Embodiments.

### 5.1. Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991).

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As used herein, the term “cell population” refers to a group of at least two cells expressing similar or different phenotypes. In non-limiting examples, a cell population can include at least about 10, at least about 100, at least about 200, at least about 300, at least about 400, at least about 500, at least about 600, at least about 700, at least about 800, at least about 900, at least about 1000 cells expressing similar or different phenotypes.

As used herein, the term “vector” refers to any genetic element, such as a plasmid, phage, transposon, cosmid, chromosome, virus, virion, etc., which is capable of replication when associated with the proper control elements and which can transfer gene sequences into cells. Thus, the term includes cloning and expression vehicles, as well as viral vectors and plasmid vectors.

As used herein, the term “expression vector” refers to a recombinant nucleic acid sequence, e.g., a recombinant DNA molecule, containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

As used herein, “CDRs” are defined as the complementarity determining region amino acid sequences of a TCR, which are the hypervariable regions of TCR α-chain and β-chain. Generally, a TCR comprises three CDRs in the α-chain variable region and three CDRs in the β-chain variable region. CDRs provide the majority of contact residues for the binding of the TCR to the antigen or epitope. CDRs regions can be delineated using the Kabat system (Kabat, E. A., et al. (1991) Sequences of Proteins of Immunological Interest, Fifth Edition, U.S. Department of Health and Human Services, NIH Publication No. 91-3242), the Chothia numbering system (Chothia et al., J Mol Biol. (1987) 196:901-17), the AbM numbering system (Abhinandan et al., Mol. Immunol. 2008, 45, 3832-3839), or the IMGT numbering system (accessible at http://www.imgt.org/IMGTScientificChart/Numbering/IMGTIGVLsuperfamily.html#table1, http://www.imgt.org/IMGTScientificChart/Nomenclature/IMGT-FRCDRdefinition.html). In certain embodiments, the CDRs regions are delineated using the IMGT numbering system.

The terms “substantially homologous” or “substantially identical” mean a polypeptide or nucleic acid molecule that exhibits at least 50% homology or identity to a reference amino acid sequence (for example, any one of the amino acid sequences described herein) or nucleic acid sequence (for example, any one of the nucleic acid sequences described herein). For example, such a sequence is at least about 60%, about 65%, about 70%, about 75%, about 80%, about 85%, about 90%, about 95% or even about 99% homologous or identical at the amino acid level or nucleic acid to the sequence used for comparison.

Sequence homology or sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. In an exemplary approach to determining the degree of identity, a BLAST program may be used, with a probability score between e⁻³ and ^{e-100} indicating a closely related sequence.

As used herein, the percent homology between two amino acid sequences is equivalent to the percent identity between the two sequences. The percent identity between the two sequences is a function of the number of identical positions shared by the sequences (i.e., % homology=# of identical positions/total # of positions ×100), taking into account the number of gaps, and the length of each gap, which need to be introduced for optimal alignment of the two sequences. The comparison of sequences and determination of percent identity between two sequences can be accomplished using a mathematical algorithm.

The percent homology between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the amino acids sequences of the presently disclosed subject matter can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the specified sequences disclosed herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used.

As used herein, the term “a conservative sequence modification” refers to an amino acid modification that does not significantly affect or alter the binding characteristics of the presently disclosed TCR comprising the amino acid sequence. Conservative modifications can include amino acid substitutions, additions and deletions. Amino acids can be classified into groups according to their physicochemical properties such as charge and polarity. Conservative amino acid substitutions are ones in which the amino acid residue is replaced with an amino acid within the same group. For example, amino acids can be classified by charge: positively-charged amino acids include lysine, arginine, histidine, negatively-charged amino acids include aspartic acid, glutamic acid, neutral charge amino acids include alanine, asparagine, cysteine, glutamine, glycine, isoleucine, leucine, methionine, phenylalanine, proline, serine, threonine, tryptophan, tyrosine, and valine. In addition, amino acids can be classified by polarity: polar amino acids include arginine (basic polar), asparagine, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine. Thus, one or more amino acid residues within a CDR region can be replaced with other amino acid residues from the same group and the altered TCR can be tested for retained function (i.e., the functions set forth in (c) through (1) above) using the functional assays described herein. In certain embodiments, no more than one, no more than two, no more than three, no more than four, no more than five residues within a specified sequence or a CDR region are altered.

As used herein, the term “disease” refers to any condition or disorder that damages or interferes with the normal function of a cell, tissue, or organ. Examples of diseases include neoplasm or pathogen infection of a cell.

An “effective amount” (or “therapeutically effective amount”) is an amount sufficient to affect a beneficial or desired clinical result upon treatment. An effective amount can be administered to a subject in one or more doses. In terms of treatment, an effective amount is an amount that is sufficient to palliate, ameliorate, stabilize, reverse or slow the progression of the disease (e.g., a tumor), prevent or delay the recurrence of a tumor, or otherwise reduce the pathological consequences of the disease (e.g., a tumor). The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the immunoresponsive cells administered.

As used herein, the term “tumor” refers to an abnormal mass of tissue that forms when cells grow and divide more than they should or do not die when they should. Tumors include benign tumors and malignant tumors (known as “cancers”). Benign tumors may grow large but do not spread into, or invade, nearby tissues or other parts of the body. Malignant tumors can spread into, or invade, nearby tissues. They can also spread to other parts of the body through the blood and lymph systems. Tumor is also called neoplasm. In certain embodiments, the tumor is cancer.

As used herein, the term “immunoresponsive cell” refers to a cell that functions in an immune response or a progenitor, or progeny thereof.

As used herein, the term “modulate” refers positively or negatively alter. Exemplary modulations include an about 1%, about 2%, about 5%, about 10%, about 25%, about 50%, about 75%, or about 100% change.

As used herein, the term “increase” refers to alter positively by at least about 5%, including, but not limited to, alter positively by about 5%, by about 10%, by about 25%, by about 30%, by about 50%, by about 75%, or by about 100%.

As used herein, the term “reduce” refers to alter negatively by at least about 5% including, but not limited to, alter negatively by about 5%, by about 10%, by about 25%, by about 30%, by about 50%, by about 75%, or by about 100%.

As used herein, the term “isolated,” “purified,” or “biologically pure” refers to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or polypeptide of the presently disclosed subject matter is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

As used herein, the term “isolated cell” refers to a cell that is separated from the molecular and/or cellular components that naturally accompany the cell.

As used herein, the term “treating” or “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

An “individual” or “subject” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters, guinea pigs, rabbits, dogs, cats, sheep, pigs, goats, cattle, horses; and non-human primates such as apes and monkeys.

As used herein, the terms “recombinant T cell receptor” or “recombinant TCR” refer to a T cell receptor wherein the exact amino acid sequence of the TCR is not naturally found in a given organism (e.g., a TCR from a mammal). In certain embodiments, this term can refer to a TCR including at least one amino acid residues not found in a naturally occurring TCR. For example, but without any limitation, a recombinant TCR can have a variable chain or a constant chain including an amino acid residue that is not found in a naturally occurring TCR. In an exemplary embodiment, a recombinant TCR can have an α variable chain or a β variable chain including an amino acid residue that is not found in a naturally occurring TCR. In another exemplary embodiment, a recombinant TCR can have an α constant chain or a β constant chain including an amino acid residue that is not found in a naturally occurring TCR.

As used herein, the term “neoantigens” or “NeoAgs” refers to peptides derived from the protein products of somatic mutations found in a patient and presented by a patient's complement of human leukocyte antigen (HLA) molecules. As used herein, the term “public neoantigen” refers to neoantigen derived from a common hotspot mutation seen in multiple patients as opposed to a “private neoantigen” which is exclusive to an individual patient.

### 5.2. RAS

RAS is a family of oncoproteins encoding small GTPases involved in regulating cell growth, differentiation and survival of cells. In humans, the RAS family includes HRAS, NRAS, and KRAS. The KRAS gene has two splice variants, KRAS4A and KRAS4B. The expression of all isoforms is nearly ubiquitous, although they show quantitative and qualitative differences in expression depending on the tissue and/or developmental stage.

RAS proteins contain two domains: a G domain that binds guanosine nucleotides, and a C-terminal hypervariable region. The G domain is highly conserved between HRAS, NRAS, KRAS4A and KRAS4B and is responsible for binding and hydrolysis of guanine nucleotides. The hypervariable regions undergo differential post-translational modifications that in turn direct isoform-specific subcellular organization. RAS proteins act as binary molecular switches and cycle between an inactive GDP-bound and active GTP-bound state. Upon activation, RAS proteins recruit and activate proteins like c-Raf and PI3-kinase that result in cell proliferation, migration and protection from apoptosis.

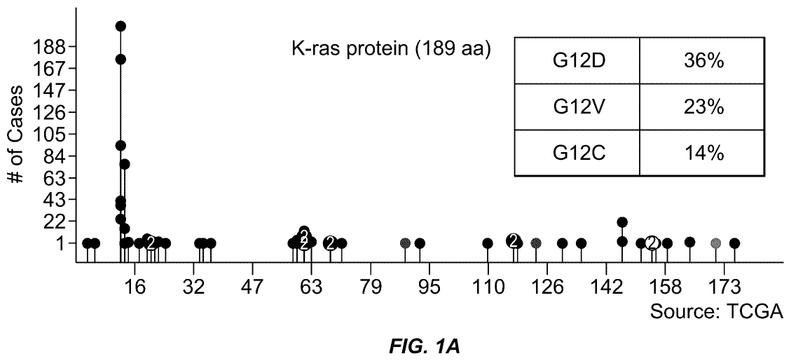

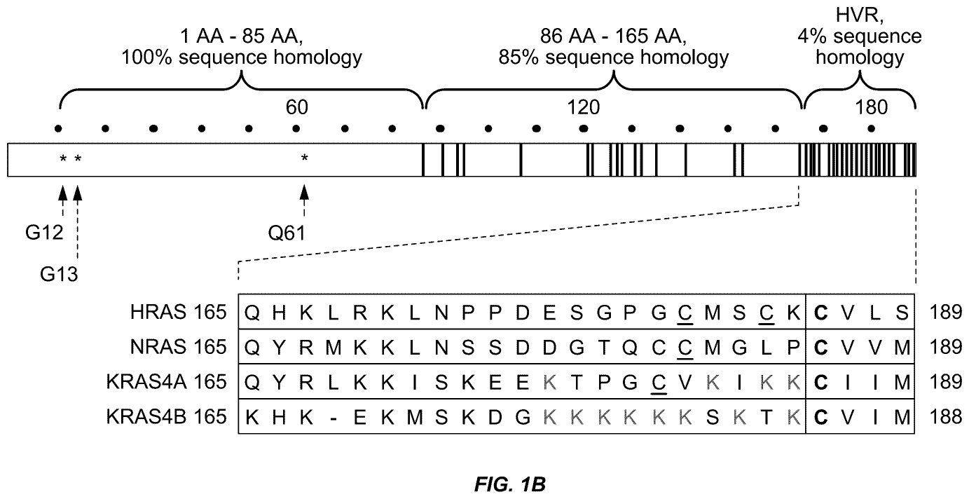

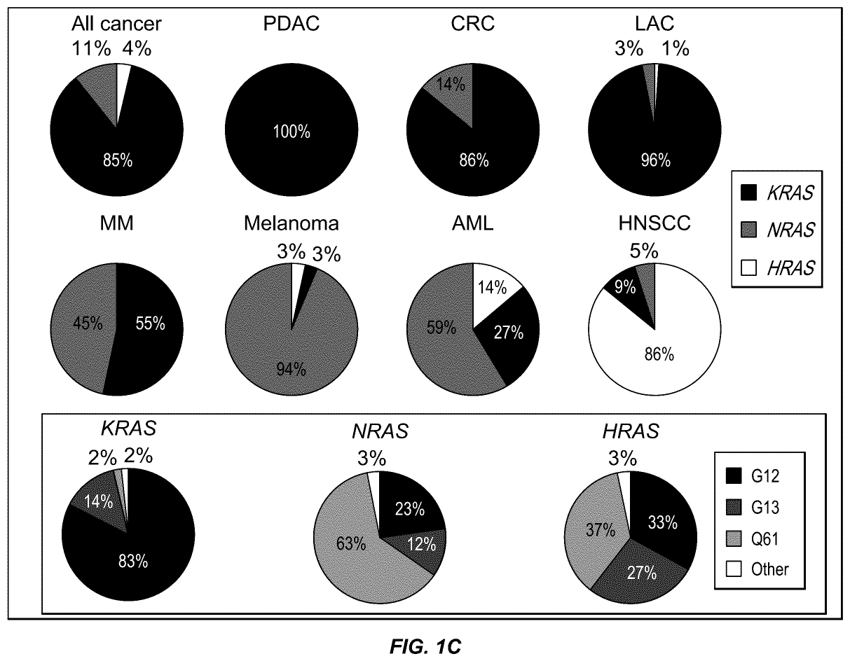

RAS mutations play a critical role in driving some of the most common and deadly carcinomas, including pancreatic, lung, and colorectal cancers, among numerous others. The conserved G domain includes several locations for hotspot mutations including G12, G13, and Q61. One of the most frequent mutations of RAS genes occur at codon 12 (i.e., G12D/V/C) (see FIG. 1). Across cancers, the most common KRAS mutation is G12D, which is a single point mutation with a glycine-to-aspartic acid substitution at codon 12. The second most common KRAS mutation is G12V, which is a single point mutation with a glycine-to-valine substitution at codon 12. The third most common KRAS mutation is G12C, which is a single point mutation with a glycine-to-cysteine substitution at codon 12.

### 5.3. T-Cell Receptor (TCR)

A TCR is a disulfide-linked heterodimeric protein consisting of two variable chains expressed as part of a non-covalent complex with the invariant CD3 chain molecules (CD3δ, CD3ε, CD3γ, CD3ζ). A TCR is found on the surface of T cells, and is responsible for recognizing antigens bound to major histocompatibility complex (MHC) molecules. In certain embodiments, a TCR comprises an α chain and a β chain (encoded by TRA and TRB, respectively). In certain embodiments, a TCR comprises a γ chain and a δ chain (encoded by TRG and TRD, respectively).

Each chain of a TCR comprises two extracellular domains: a variable region and a constant region. The constant region is proximal to the cell membrane, followed by a transmembrane domain and a short cytoplasmic tail (i.e., an intracellular domain). The variable region binds to the peptide/MHC complex. The variable region of both chains each has three complementarity determining regions (CDRs).

In certain embodiments, a TCR can form a receptor complex with three dimeric signaling modules CD3δ/ε, CD3γ/ε and CD247 ζ/ζ or ζ/η. When a TCR complex engages with its cognate peptide antigen/MHC (peptide/MHC), the T cell expressing the TCR complex is activated.

The presently disclosed subject matter provides recombinant TCRs. In certain embodiments, the recombinant TCR differs from any naturally occurring TCR by at least one amino acid residue. In certain embodiments, the recombinant TCR differs from any naturally occurring TCR by at least 2, 3, 4, 5, 6, 7, 8, 9, 10, 11, 12, 13, 14, 15, 20, 25, 30, 40, 50, 60, 70, 80, 90, 100 or more amino acid residues. In certain embodiments, the recombinant TCR is modified from a naturally occurring TCR by at least one amino acid residue. In certain embodiments, the recombinant TCR is modified from a naturally occurring TCR by at least 2, 3, 4, 5, 6, 7, 8, 9, 10, 11, 12, 13, 14, 15, 20, 25, 30, 40, 50, 60, 70, 80, 90, 100 or more amino acid residues.

In certain embodiments, the presently disclosed TCR targets or binds to a RAS peptide that comprises a mutation (“a mutant RAS peptide”). In certain embodiments, the mutation is a point mutation. In certain embodiments, the mutation is a G12 mutation. In certain embodiments, the RAS peptide comprises or consists of the amino acid sequence set forth in SEQ ID NO: 1. In certain embodiments, the RAS peptide comprises or consists of the amino acid sequence set forth in SEQ ID NO: 2. In certain embodiments, the presently disclosed TCR does not bind to a wildtype RAS. In certain embodiments, the presently disclosed TCR binds to a RAS peptide comprising or consisting of the amino acid sequence set forth in SEQ ID NO: 3. In certain embodiments, the RAS peptide comprises or consists of the amino acid sequence set forth in SEQ ID NO: 4. In certain embodiments, the RAS peptide comprises or consists of the amino acid sequence set forth in SEQ ID NO: 5. In certain embodiments, the RAS peptide comprises or consists of the amino acid sequence set forth in SEQ ID NO: 6. SEQ ID NOs: 1-6 are provided below.

[SEQ ID NO: 1]
VVGADGVGK
[SEQ ID NO: 2]
VVVGADGVGK
[SEQ ID NO: 3]
VVVGAGGVGK
[SEQ ID NO: 4]
VVGAVGVGK
[SEQ ID NO: 5]
VVVGAVGVGK
[SEQ ID NO: 6]
VVVGACGVGK

In certain embodiments, the presently disclosed TCR targets or binds to KRAS comprising a RAS peptide comprising or consisting of the amino acid sequence set forth in SEQ ID NO: 1. In certain embodiments, the presently disclosed TCR targets or binds to KRAS comprising a RAS peptide comprising or consisting of the amino acid sequence set forth in SEQ ID NO: 2. In certain embodiments, the presently disclosed TCR targets or binds to KRAS comprising a RAS peptide comprising or consisting of the amino acid sequence set forth in SEQ ID NO: 3. In certain embodiments, the presently disclosed TCR targets or binds to KRAS comprising a RAS peptide comprising or consisting of the amino acid sequence set forth in SEQ ID NO: 4. In certain embodiments, the presently disclosed TCR targets or binds to KRAS comprising a RAS peptide comprising or consisting of the amino acid sequence set forth in SEQ ID NO: 5. In certain embodiments, the presently disclosed TCR targets or binds to KRAS comprising a RAS peptide comprising or consisting of the amino acid sequence set forth in SEQ ID NO: 6.

In certain embodiments, the presently disclosed TCR targets or binds to NRAS comprising a RAS peptide comprising or consisting of the amino acid sequence set forth in SEQ ID NO: 1. In certain embodiments, the presently disclosed TCR targets or binds to NRAS comprising a RAS peptide comprising or consisting of the amino acid sequence set forth in SEQ ID NO: 2. In certain embodiments, the presently disclosed TCR targets or binds to NRAS comprising a RAS peptide comprising or consisting of the amino acid sequence set forth in SEQ ID NO: 3. In certain embodiments, the presently disclosed TCR targets or binds to NRAS comprising a RAS peptide comprising or consisting of the amino acid sequence set forth in SEQ ID NO: 4. In certain embodiments, the presently disclosed TCR targets or binds to NRAS comprising a RAS peptide comprising or consisting of the amino acid sequence set forth in SEQ ID NO: 5. In certain embodiments, the presently disclosed TCR targets or binds to NRAS comprising a RAS peptide comprising or consisting of the amino acid sequence set forth in SEQ ID NO: 6.

In certain embodiments, the presently disclosed TCR targets or binds to HRAS comprising a RAS peptide comprising or consisting of the amino acid sequence set forth in SEQ ID NO: 1. In certain embodiments, the presently disclosed TCR targets or binds to HRAS comprising a RAS peptide comprising or consisting of the amino acid sequence set forth in SEQ ID NO: 2. In certain embodiments, the presently disclosed TCR targets or binds to HRAS comprising a RAS peptide comprising or consisting of the amino acid sequence set forth in SEQ ID NO: 3. In certain embodiments, the presently disclosed TCR targets or binds to HRAS comprising a RAS peptide comprising or consisting of the amino acid sequence set forth in SEQ ID NO: 4. In certain embodiments, the presently disclosed TCR targets or binds to HRAS comprising a RAS peptide comprising or consisting of the amino acid sequence set forth in SEQ ID NO: 5. In certain embodiments, the presently disclosed TCR targets or binds to HRAS comprising a RAS peptide comprising or consisting of the amino acid sequence set forth in SEQ ID NO: 6.

In certain embodiments, the presently disclosed TCR targets or binds to a RAS peptide associated with an HLA class I complex, e.g., HLA-A, HLA-B and HLA-C.

In certain embodiments, the presently disclosed TCR targets or binds to a RAS peptide associated with an HLA-A*03 superfamily (e.g., in an HLA-A*03 superfamily dependent manner). In certain embodiments, the HLA*A03 superfamily members, include, but not limited to, alleles and sub-alleles in the HLA-A*03, HLA-A*11, HLA-A*31, HLA-A*33, HLA-A*66, HLA-A*68 and HLA-A*74. In certain embodiments, the presently disclosed TCR targets or binds to a RAS peptide associated with an HLA-A*11 molecule.

### 5.3.1. TCRs

### 5.3.1.1. Variable Regions

In certain embodiments, the extracellular domain of the TCR comprises an α chain variable region comprising a CDR1, a CDR2, and a CDR3. In certain embodiments, the extracellular domain of the TCR comprises an α chain variable region comprising a CDR1 comprising the amino acid sequence set forth in SEQ ID NO: 16 or a conservative modification thereof, a CDR2 comprising the amino acid sequence set forth in SEQ ID NO: 17 or a conservative modification thereof, and a CDR3 comprising the amino acid sequence set forth in SEQ ID NO: 18 or a conservative modification thereof. In certain embodiments, the α chain variable region comprises a CDR1 comprising the amino acid sequence set forth in SEQ ID NO: 16, a CDR2 comprising the amino acid sequence set forth in SEQ ID NO: 17, and a CDR3 comprising the amino acid sequence set forth in SEQ ID NO: 18. SEQ ID NOs: 16-18 are provided below:

[SEQ ID NO: 16]
SIFNT
[SEQ ID NO: 17]
LYKAGEL
[SEQ ID NO: 18]
CAGRREGAQKLVF

In certain embodiments, the extracellular domain of the TCR comprises a β chain variable region comprising a CDR1, a CDR2, and a CDR3. In certain embodiments, the extracellular domain of the TCR comprises an β chain variable region comprising a CDR1 comprising the amino acid sequence set forth in SEQ ID NO: 19 or a conservative modification thereof, a CDR2 comprising the amino acid sequence set forth in SEQ ID NO: 20 or a conservative modification thereof, and a CDR3 comprising the amino acid sequence set forth in SEQ ID NO: 21 or a conservative modification thereof. In certain embodiments, the β chain variable region comprises a CDR1 comprising the amino acid sequence set forth in SEQ ID NO: 19, a CDR2 comprising the amino acid sequence set forth in SEQ ID NO: 20, and a CDR3 comprising the amino acid sequence set forth in SEQ ID NO: 21. SEQ ID NOs: 19-21 are provided below:

[SEQ ID NO: 19]
SGHVS
[SEQ ID NO: 20]
FQNEAQ
[SEQ ID NO: 21]
CASSSTLMGVNIQYF

In certain embodiments, the α chain variable region comprises a CDR1 comprising the amino acid sequence set forth in SEQ ID NO: 16 or a conservative modification thereof, a CDR2 comprising the amino acid sequence set forth in SEQ ID NO: 17 or a conservative modification thereof, and a CDR3 comprising the amino acid sequence set forth in SEQ ID NO: 18 or a conservative modification thereof, and the β chain variable region comprises a CDR1 comprising the amino acid sequence set forth in SEQ ID NO: 19 or a conservative modification thereof, a CDR2 comprising the amino acid sequence set forth in SEQ ID NO: 20 or a conservative modification thereof, and a CDR3 comprising the amino acid sequence set forth in SEQ ID NO: 21 or a conservative modification thereof. In certain embodiments, the α chain variable region comprises a CDR1 comprising the amino acid sequence set forth in SEQ ID NO: 16, a CDR2 comprising the amino acid sequence set forth in SEQ ID NO: 17, and a CDR3 comprising the amino acid sequence set forth in SEQ ID NO: 18; and the β chain variable region comprises a CDR1 comprising the amino acid sequence set forth in SEQ ID NO: 19, a CDR2 comprising the amino acid sequence set forth in SEQ ID NO: 20, and a CDR3 comprising the amino acid sequence set forth in SEQ ID NO: 21. In certain embodiments, the CDRs sequences described above including are delineated using the IMGT numbering system.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 4. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 7. In certain embodiments, the α chain variable region comprises the amino acid sequence set forth in SEQ ID NO: 7. SEQ ID NO: 7 is provided below:

[SEQ ID NO: 7]
MLLEHLLIILWMQLTWVSGQQLNQSPQSMFIQEGEDVSMNCTSSSIFNTW
LWYKQDPGEGPVLLIALYKAGELTSNGRLTAQFGITRKDSFLNISASIPS
DVGIYFCAGRREGAQKLVFGQGTRLTINP

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 8. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 8. In certain embodiments, the β chain variable region comprises the amino acid sequence set forth in SEQ ID NO: 8. SEQ ID NO: 8 is provided below:

[SEQ ID NO: 8]
MGTRLLCWVVLGFLGTDHTGAGVSQSPRYKVAKRGQDVALRCDPISGHVS
LFWYQQALGQGPEFLTYFQNEAQLDKSGLPSDRFFAERPEGSVSTLKIQR
TQQEDSAVYLCASSSTLMGVNIQYFGAGTRLSVL

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 7; and the β chain variable region comprises an amino acid sequence that is at least about 80% (e.g., at least about 85%, at least about 90%, or at least about 95%) homologous or identical to the amino acid sequence set forth in SEQ ID NO: 8. In certain embodiments, the α chain variable region comprises the amino acid sequence set forth in SEQ ID NO: 7; and the β chain variable region comprises the amino acid sequence set forth in SEQ ID NO: 8.

In certain embodiments, the TCR is designated as “TCR T3”. In certain embodiments, the TCR T3 binds to a RAS peptide comprising or consisting of the amino acid sequence set forth in SEQ ID NO: 1. In certain embodiments, the TCR T3 binds to a RAS peptide comprising or consisting of the amino acid sequence set forth in SEQ ID NO: 2. In certain embodiments, the TCR T3 binds to a RAS peptide comprising or consisting of the amino acid sequence set forth in SEQ ID NO: 3. In certain embodiments, the TCR T3 binds to a RAS peptide comprising or consisting of the amino acid sequence set forth in SEQ ID NO: 4. In certain embodiments, the TCR T3 binds to a RAS peptide comprising or consisting of the amino acid sequence set forth in SEQ ID NO: 5. In certain embodiments, the TCR T3 binds to a RAS peptide comprising or consisting of the amino acid sequence set forth in SEQ ID NO: 6.

In certain embodiments, the α chain variable region and/or the β chain variable region amino acid sequences having at least about 80%, at least about 85%, at least about 90%, or at least about 95% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homology or identity to the specified sequences (e.g., SEQ ID NO: 7 and SEQ ID NO: 8) comprise modifications, including, but not limited to, substitutions (e.g., conservative substitutions), insertions, or deletions relative to the specified sequence(s), but retain the ability to bind to a mutant RAS peptide (e.g., a G12D mutant RAS peptide). In certain embodiments, such modifications are not within the CDR domains of the variable regions.

In certain embodiments, a total of 1 to 10 amino acids are substituted, inserted and/or deleted in SEQ ID NO: 7 or SEQ ID NO: 8. In certain embodiments, substitutions, insertions, or deletions occur in regions outside the CDRs of the extracellular domain. In certain embodiments, the extracellular domain comprises an α chain variable region and/or a β chain variable region set forth in SEQ ID NO: 7 and SEQ ID NO: 8, including post-translational modifications of that sequence (SEQ ID NO: 7 or SEQ ID NO: 8).

### 5.3.1.2. Constant Regions

In certain embodiments, the presently disclosed TCR comprises an α chain constant region that comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 9, SEQ ID NO: 10, or SEQ ID NO: 25. In certain embodiments, the α chain constant region comprises the amino acid sequence set forth in SEQ ID NO: 9. In certain embodiments, the α chain constant region comprises the amino acid sequence set forth in SEQ ID NO: 10. In certain embodiments, the α chain constant region comprises the amino acid sequence set forth in SEQ ID NO: 25.

In certain embodiments, the presently disclosed TCR comprises a β chain constant region that comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 11, SEQ ID NO: 12, SEQ ID NO: 13, or SEQ ID NO: 14. In certain embodiments, the β chain constant region comprises the amino acid sequence set forth in SEQ ID NO: 11. In certain embodiments, the β chain constant region comprises the amino acid sequence set forth in SEQ ID NO: 12. In certain embodiments, the β chain constant region comprises the amino acid sequence set forth in SEQ ID NO: 13. In certain embodiments, the β chain constant region comprises the amino acid sequence set forth in SEQ ID NO: 14. SEQ ID NOS: 9-14 are provided below:

Human α chain constant region:

[SEQ ID NO: 9]
NIQNPDPAVYQLRDSKSSDKSVCLFTDFDSQTNVSQSKDSDVYITDKTVL
DMRSMDFKSNSAVAWSNKSDFACANAFNNSIIPEDTFFPSPESSCDVKLV
EKSFETDTNLNFQNLSVIGFRILLLKVAGFNLLMTLRLWSS

Mouse α chain constant region (cysteine-modification and LVL modification in transmembrane domain underlined):

[SEQ ID NO: 10]
NIQNPEPAVYQLKDPRSQDSTLCLFTDFDSQINVPKTMESGTFITDKCVL
DMKAMDSKSNGAIAWSNQTSFTCQDIFKETNATYPSSDVPCDATLTEKSF
ETDMNLNFQNLLVIVLRILLLKVAGFNLLMTLRLWSS

Mouse α chain constant region (native):

[SEQ ID NO: 25]
DIQNPEPAVYQLKDPRSQDSTLCLFTDFDSQINVPKTMESGTFITDKTVL
DMKAMDSKSNGAIAWSNQTSFTCQDIFKETNATYPSSDVPCDATLTEKSF
ETDMNLNFQNLSVMGLRILLLKVAGFNLLMTLRL

Human β chain constant region:

[SEQ ID NO: 11]
EDLNKVFPPEVAVFEPSEAEISHTQKATLVCLATGFFPDHVELSWWVNGK
EVHSGVSTDPQPLKEQPALNDSRYCLSSRLRVSATFWQNPRNHFRCQVQF
YGLSENDEWTQDRAKPVTQIVSAEAWGRADCGFTSVSYQQGVLSATILYE
ILLGKATLYAVLVSALVLMAMVKRKDF

Human β chain constant region:

[SEQ ID NO: 12]
EDLKNVFPPKVAVFEPSEAEISHTQKATLVCLATGFYPDHVELSWWVNGK
EVHSGVSTDPQPLKEQPALNDSRYCLSSRLRVSATFWQNPRNHFRCQVQF
YGLSENDEWTQDRAKPVTQIVSAEAWGRADCGFTSESYQQGVLSATILYE
ILLGKATLYAVLVSALVLMAMVKRKDSRG

Human β chain constant region:

[SEQ ID NO: 13]
EDLKNVFPPEVAVFEPSEAEISHTQKATLVCLATGFYPDHVELSWWVNGK
EVHSGVSTDPQPLKEQPALNDSRYCLSSRLRVSATFWQNPRNHFRCQVQF
YGLSENDEWTQDRAKPVTQIVSAEAWGRADCGFTSESYQQGVLSATILYE
ILLGKATLYAVLVSALVLMAMVKRKDSRG

Mouse β chain constant region (cysteine-modification underlined):

[SEQ ID NO: 14]
EDLRNVTPPKVSLFEPSKAEIANKQKATLVCLARGFFPDHVELSWWVNGK
EVHSGVCTDPQAYKESNYSYCLSSRLRVSATFWHNPRNHFRCQVQFHGLS
EEDKWPEGSPKPVTQNISAEAWGRADCGITSASYQQGVLSATILYEILLG
KATLYAVLVSTLVVMAMVKRKNS

5.3.2. TCRs that Bind to the Same RAS Peptide as TCR Clonotypes

The presently disclosed subject matter further provides TCRs that bind to the same RAS peptide (e.g., a G12D mutant RAS peptide) as a TCR disclosed herein (e.g., a TCR disclosed in Section 5.3.1). In certain embodiments, the TCR binds to the same RAS peptide (e.g., a G12D mutant RAS peptide) as a reference TCR or a functional fragment thereof comprising the α chain variable region comprising a CDR1, a CDR2, and a CDR3 and the β chain variable region comprising a CDR1, a CDR2, and a CDR3 of, for example, the TCRs disclosed herein (e.g., disclosed in Section 5.3.1). In certain embodiments, the TCR binds to the same RAS peptide (e.g., a G12D mutant RAS peptide) as a reference TCR or a functional fragment thereof comprising the α chain variable region and the β chain variable region sequences of, for example, the presently disclosed TCR (e.g., disclosed in Section 5.3.1).

### 5.3.3. TCRs Having Specific CDR3 Sequences

It is well known in the art that the CDR3 domain, independently from the CDR1 and/or CDR2 domain(s), alone can determine the binding specificity of a TCR or a functional fragment thereof, for a cognate antigen and that multiple TCRs can predictably be generated having the same binding specificity based on a common CDR3 sequence.

In certain embodiments, the extracellular domain of the TCR comprises an α chain variable region CDR3 comprising the amino acid sequence set forth in SEQ ID NO: 18 or a conservative modification thereof; and a β chain variable region CDR3 comprising the amino acid sequence set forth in SEQ ID NO: 21 or a conservative modification thereof. In certain embodiments, the extracellular domain of the TCR further comprises an α chain variable region CDR2 comprising the amino acid sequence set forth in SEQ ID NO: 17 or a conservative modification thereof; and a β chain variable region CDR2 comprising the amino acid sequence set forth in SEQ ID NO: 20 or a conservative modification thereof. In certain embodiments, the extracellular domain of the TCR further comprises an α chain variable region CDR1 comprising the amino acid sequence set forth in SEQ ID NO: 16 or a conservative modification thereof; and a β chain variable region CDR1 comprising the amino acid sequence set forth in SEQ ID NO: 19 or a conservative modification thereof.

5.3.4. TCRs with Modifications within CDRs

In certain embodiments, a presently disclosed TCR (or a functional fragment thereof) comprises an α chain variable region comprising a CDR1, a CDR2 and a CDR3 sequences and a β chain variable region comprising a CDR1, a CDR2 and a CDR3 sequences, wherein one or more of these CDR sequences comprise specified amino acid sequences based on the TCR (or a functional fragments thereof) described herein, or modifications thereof, and wherein the TCR (or a functional fragments thereof) retains the desired functional properties of the mutant RAS peptide-specific TCR (or a functional fragments thereof) of the presently disclosed subject matter.

In certain embodiments, a presently disclosed TCR (or a functional fragment thereof) comprises an α chain constant region and a β chain constant region, wherein at least one of the constant regions comprises specified amino acid sequences based on the TCR (or a functional fragments thereof) described herein, or modifications thereof, and wherein the TCR (or a functional fragment thereof) retains the desired functional properties of the mutant RAS peptide-specific TCRs (or a functional fragments thereof) of the presently disclosed subject matter.

In certain embodiments, such modifications do not significantly affect or alter the binding characteristics of the TCR comprising the amino acid sequence. Non-limiting examples of such modifications include amino acid substitutions, additions and deletions. Modifications can be introduced into the presently disclosed TCR or a functional fragment thereof by standard techniques known in the art, such as site-directed mutagenesis and PCR-mediated mutagenesis.

The modifications can be conservative modifications, non-conservative modifications, or mixtures of conservative and non-conservative modifications. As discussed above, conservative amino acid substitutions are ones in which the amino acid residue is replaced with an amino acid residue having a similar side chain. Families of amino acid residues having similar side chains have been defined in the art. Exemplary conservative amino acid substitutions are shown in Table 1. In certain embodiments, amino acid substitutions may be introduced into a TCR of interest and the products screened for a desired activity, e.g., retained/improved antigen binding, decreased immunogenicity, or improved ADCC or CDC.

TABLE 1

| | Exemplary conservative |
|---|---|
| Original Residue | amino acid Substitutions |
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe |
| Leu (L) | Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala |

Amino acids may be grouped according to common side-chain properties:
hydrophobic: Norleucine, Met, Ala, Val, Leu, Ile;
neutral hydrophilic: Cys, Ser, Thr, Asn, Gln;
acidic: Asp, Glu;
basic: His, Lys, Arg;
residues that influence chain orientation: Gly, Pro;
aromatic: Trp, Tyr, Phe.

In certain embodiments, one or more amino acid residues within a CDR region can be replaced with other amino acid residues from the same group and the altered TCR can be tested for retained function using the functional assays described herein.

Non-conservative substitutions entail exchanging a member of one of these classes for another class.

In certain embodiments, no more than one, no more than two, no more than three, no more than four, no more than five residues within a specified sequence or a CDR region are altered.

In certain embodiments, one or more amino acid residues within a constant region of a TCR can be modified to enhance stability and/or cell surface expression of the TCR. In certain embodiments, no more than one, no more than two, no more than three, no more than four, no more than five residues within a specified sequence or a constant region are altered. In certain embodiments, the modification includes but is not limited to, murinization, cysteine modification and transmembrane modification (see Cohen et al. Enhanced antitumor activity of murine-human hybrid T-cell receptor (TCR) in human lymphocytes is associated with improved pairing and TCR/CD3 stability, Cancer Res. 2006; 66(17):8878-8886; Cohen et al. Enhanced antitumor activity of T cells engineered to express T-cell receptors with a second disulfide bond, Cancer Res. 2007; 67(8):3898-3903; Kuball et al. Facilitating matched pairing and expression of TCR chains introduced into human T cells, Blood 2007; 109(6):2331-2338; Haga-Friedman et al. Incorporation of transmembrane hydrophobic mutations in the TCR enhance its surface expression and T cell functional avidity, Journal of immunology 2012; 188(11):5538-5546, the contents of each of which are incorporated by reference in their entireties).

### 5.3.5. Multispecific Molecules

The presently disclosed subject matter provides bispecific molecules comprising a presently disclosed TCR (or a functional fragment thereof). Additionally or alternatively, the presently disclosed subject matter provides bispecific molecules comprising a presently disclosed TCR (or a functional fragment thereof). A presently disclosed TCR or a functional fragment thereof can be derivatized or linked to another functional molecule, e.g., another peptide or protein (e.g., another antibody or ligand for a receptor) to generate a multispecific molecule (e.g., a bispecific molecule) that binds to at least two different binding sites or target molecules. The presently disclosed TCR or a functional fragment thereof can in fact be derivatized or linked to more than one other functional molecule to generate multi-specific molecules that bind to more than two different binding sites and/or target molecules; such multi-specific molecules are also intended to be encompassed by the term “multispecific molecule” or “bispecific molecule” as used herein. To create a multispecific molecule (e.g., a bispecific molecule), a presently disclosed TCR or a functional fragment thereof can be functionally linked (e.g., by chemical coupling, genetic fusion, noncovalent association or otherwise) to one or more other binding molecules, such as another antibody, antibody fragment, peptide or binding mimetic.

The presently disclosed subject matter provides multispecific molecules comprising at least a first binding specificity for a mutant RAS peptide and a second binding specificity for a second target peptide region. The second target epitope region can be a second RAS peptide, or a non-RAS peptide, e.g., a different antigen. In certain embodiments, the multi-specific molecule further comprises a third binding specificity. Where a first portion of a multispecific molecule, e.g., antibody, binds to an antigen on a tumor cell for example and a second portion of a multispecific molecule recognizes an antigen on the surface of a human immune effector cell, the multispecific molecule is capable of recruiting the activity of that effector cell by specifically binding to the effector antigen on the human immune effector cell. In certain embodiments, multispecific molecules are able to form a link between effector cells, for example, T cells and tumor cells, thereby enhancing effector function. In certain embodiments, a presently disclosed multispecific molecule comprises at least a first binding to a mutant RAS peptide and at least a second binding to an immune cell or a molecule associated with an immune cell.

The presently disclosed subject matter provides bispecific molecules comprising at least a first binding specificity for a mutant RAS peptide and a second binding specificity for a second target peptide region. The second target epitope region can be a second RAS peptide, or a non-RAS peptide, e.g., a different antigen. In certain embodiments, the bispecific molecule is multi-specific, e.g., the molecule can further include a third binding specificity. Where a first portion of a bispecific molecule, e.g., antibody, binds to an antigen on a tumor cell for example and a second portion of a bispecific molecule recognizes an antigen on the surface of a human immune effector cell, the bispecific molecule is capable of recruiting the activity of that effector cell by specifically binding to the effector antigen on the human immune effector cell. In certain embodiments, bispecific molecules are able to form a link between effector cells, for example, T cells and tumor cells, thereby enhancing effector function. In certain embodiments, a presently disclosed bispecific molecule comprises at least a first binding to a mutant RAS peptide and at least a second binding to an immune cell or a molecule associated with an immune cell.

The multispecific molecules and bispecific molecules of the presently disclosed subject matter can be prepared by conjugating the constituent binding specificities using methods known in the art. For example, each binding specificity of the bispecific molecule can be generated separately and then conjugated to one another. When the binding specificities are proteins or peptides, a variety of coupling or cross-linking agents can be used for covalent conjugation. Non-limiting examples of cross-linking agents include protein A, carbodiimide, N-succinimidyl-S-acetyl-thioacetate (SATA), 5, 5′-dithiobis(2-nitrobenzoic acid) (DTNB), o-phenylenedimaleimide (oPDM), N-succinimidyl-3-(2-pyridyldithio)propionate (SPDP), and sulfosuccinimidyl 4-(N-maleimidomethyl) cyclohaxane-1-carboxylate (sulfo-SMCC) (see e.g., Karpovsky et al. (1984) J. Exp. Med. 160:1686; Liu, M A et al. (1985) Proc. Natl. Acad. Sci. USA 82:8648). Other methods include those described in Paulus (1985) Behring Ins. Mitt. No. 78, 118-132; Brennan et al. (1985) Science 229:81-83), and Glennie et al. (1987) J. Immunol. 139: 2367-2375). Conjugating agents can be SATA and sulfo-SMCC, both available from Pierce Chemical Co. (Rockford, IL).

When the binding specificities are antibodies, they can be conjugated via sulfhydryl bonding of the C-terminus hinge regions of the two heavy chains. In certain embodiments, the hinge region is modified to contain an odd number of sulfhydryl residues, preferably one, prior to conjugation.

Alternatively, both binding specificities can be encoded in the same vector and expressed and assembled in the same host cell. This method is particularly useful where the bispecific molecule is a mAb and a mAb, a mAb and a Fab, a Fab and a F(ab′)₂, or a ligand and a Fab fusion protein.

Binding of the multispecific molecules and bispecific molecules to their specific targets can be confirmed by, for example, enzyme-linked immunosorbent assay (ELISA), radioimmunoassay (RIA), FACS analysis, bioassay (e.g., growth inhibition), or Western Blot assay. Each of these assays generally detects the presence of protein-antibody complexes of particular interest by employing a labeled reagent (e.g., an antibody) specific for the complex of interest. Alternatively, the complexes can be detected using any of a variety of other immunoassays. For example, the antibody can be radioactively labeled and used in a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a γ counter or a scintillation counter or by autoradiography.

### 5.4. Cells

The presently disclosed subject matter provides cells comprising a presently disclosed TCR (e.g., one disclosed in Section 5.3). In certain embodiments, the cell is selected from the group consisting of cells of lymphoid lineage, cells of myeloid lineage, stem cells from which cells of lymphoid lineage can be derived, and stem cells from which cells of myeloid lineage can be derived. In certain embodiments, the cell is an immunoresponsive cell. In certain embodiments, the immunoresponsive cell is a cell of lymphoid lineage.

In certain embodiments, the cell is a cell of the lymphoid lineage. Cells of the lymphoid lineage can provide production of antibodies, regulation of cellular immune system, detection of foreign agents in the blood, detection of cells foreign to the host, and the like. Non-limiting examples of cells of the lymphoid lineage include T cells and/or stem cells from which lymphoid cells may be differentiated. In certain embodiments, the stem cell is a pluripotent stem cell (e.g., embryonic stem cell).

In certain embodiments, the cell is a T cell. T cells can be lymphocytes that mature in the thymus and are chiefly responsible for cell-mediated immunity. T cells are involved in the adaptive immune system. The T cells of the presently disclosed subject matter can be any type of T cells, including, but not limited to, helper T cells, cytotoxic T cells, memory T cells (including central memory T cells, stem-cell-like memory T cells (or stem-like memory T cells), and two types of effector memory T cells: e.g., TEM cells and TEMRA cells, Regulatory T cells (also known as suppressor T cells), tumor-infiltrating lymphocyte (TIL), Natural killer T cells, Mucosal associated invariant T cells, and γδ T cells. Cytotoxic T cells (CTL or killer T cells) are a subset of T lymphocytes capable of inducing the death of infected somatic or tumor cells. A patient's own T cells may be genetically modified to target specific antigens through the introduction of an antigen-recognizing receptor, e.g., a CAR. In certain embodiments, the immunoresponsive cell is a T cell. The T cell can be a CD4⁺ T cell or a CD8⁺ T cell. In certain embodiments, the T cell is a CD4⁺ T cell. In certain embodiments, the T cell is a CD8⁺ T cell. In certain embodiments, the TCR-expressing T cells express Foxp3 to achieve and maintain a T regulatory phenotype.

In certain embodiments, the T cell is a NK-T cell. Natural killer (NK) T cells can be lymphocytes that are part of cell-mediated immunity and act during the innate immune response. NK-T cells do not require prior activation in order to perform their cytotoxic effect on target cells.

In certain embodiments, the cell is an Natural Killer (NK) cell. NK cells constitute the predominant innate lymphocyte subset that physiologically mediates the anti-viral and anti-tumor immune responses. NK cells use an array of innate receptors to sense their environment and to respond to infections, cellular stress, and transformation. The resulting NK cell activation, including cytotoxicity and cytokine production, is a component of the early immune response.

Types of human lymphocytes of the presently disclosed subject matter include, without limitation, peripheral donor lymphocytes. e.g., those disclosed in Sadelain et al., Nat Rev Cancer (2003); 3:35-45 (disclosing peripheral donor lymphocytes genetically modified to express CARs), in Morgan, R. A., et al. 2006 Science 314:126-129 (disclosing peripheral donor lymphocytes genetically modified to express a full-length tumor antigen-recognizing T cell receptor complex comprising the α and β heterodimer), in Panelli et al., J Immunol (2000); 164:495-504; Panelli et al., J Immunol (2000); 164:4382-4392 (disclosing lymphocyte cultures derived from tumor infiltrating lymphocytes (TILs) in tumor biopsies), and in Dupont et al., Cancer Res (2005); 65:5417-5427; Papanicolaou et al., Blood (2003); 102:2498-2505 (disclosing selectively in vitro-expanded antigen-specific peripheral blood leukocytes employing artificial antigen-presenting cells (AAPCs) or pulsed dendritic cells).

The cells (e.g., T cells) can be autologous, non-autologous (e.g., allogeneic), or derived in vitro from engineered progenitor or stem cells.

The cells of the presently disclosed subject matter can be cells of the myeloid lineage. Non-limiting examples of cells of the myeloid lineage include monocytes, macrophages, neutrophils, dendritic cells, basophils, neutrophils, eosinophils, megakaryocytes, mast cell, erythrocyte, thrombocytes, and stem cells from which myeloid cells may be differentiated. In certain embodiments, the stem cell is a pluripotent stem cell (e.g., an embryonic stem cell or an induced pluripotent stem cell).

In certain embodiments, the cell further comprises at least one recombinant or exogenous co-receptor. For example, a presently disclosed cell can be further transduced with at least one co-receptor, such that the cell co-expresses or is induced to co-express the presently disclosed TCR and the at least one co-receptor. The interaction between the presently disclosed TCR and at least one co-receptor with the MHC complex of the target cell (e.g., tumor cell associated with a RAS mutation) improves the antigen-specific signal required for full activation of an immunoresponsive cell (e.g., T cell).

In certain embodiments, the co-receptor is a CD8 co-receptor. In certain embodiments, the CD8 co-receptor comprises an α chain and a β chain. In certain embodiments, the α chain of the CD8 co-receptor comprises or consists of an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence having a UniProt Reference No: P01732, or fragments thereof, and/or may optionally comprise up to one or up to two or up to three conservative amino acid substitutions. In certain embodiments, the α chain of the CD8 co-receptor comprises or consists of an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 22. In certain embodiments, the α chain of the CD8 co-receptor comprises or consists of the amino acid sequence set forth in SEQ ID NO: 22.

In certain embodiments, the β chain of the CD8 co-receptor comprises or consists of an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence having a UniProt Reference No: P10966, or fragments thereof, and/or may optionally comprise up to one or up to two or up to three conservative amino acid substitutions. In certain embodiments, the β chain of the CD8 co-receptor comprises or consists of an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 23. In certain embodiments, the β chain of the CD8 co-receptor comprises or consists of the amino acid sequence set forth in SEQ ID NO: 23.

In certain embodiments, the CD8 co-receptor comprises an α chain comprising or consists of an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 22; and a β chain comprising or consists of an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 23. In certain embodiments, the CD8 co-receptor comprises an α chain comprising or consists of the amino acid sequence set forth in SEQ ID NO: 22; and a β chain comprising or consists of the amino acid sequence set forth in SEQ ID NO: 23. SEQ ID NO: 22 and SEQ ID NO: 23 are provided below:

[SEQ ID NO: 22]
MALPVTALLLPLALLLHAARPSQFRVSPLDRTWNLGETVELKCQVLLSNP
PSGCSWLFQPRGAAASPTFLLYLSQNKPKAAEGLDTQRFSGKRLGDTFVL
TLSDFRRENEGYYFCSALSNSIMYFSHFVPVFLPAKPTTTPAPRPPTPAP
TIASQPLSLRPEACRPAAGGAVHTRGLDFACDIYIWAPLAGTCGVLLLSL
VITLYCNHRNRRRVCKCPRPVVKSGDKPSLSARYV
[SEQ ID NO: 23]
MRPRLWLLLAAQLTVLHGNSVLQQTPAYIKVQTNKMVMLSCEAKISLSNM
RIYWLRQRQAPSSDSHHEFLALWDSAKGTIHGEEVEQEKIAVFRDASRFI
LNLTSVKPEDSGIYFCMIVGSPELTFGKGTQLSVVDFLPTTAQPTKKSTL
KKRVCRLPRPETQKGPLCSPITLGLLVAGVLVLLVSLGVAIHLCCRRRRA
RLRFMKQFYK

In certain embodiments, the co-receptor is a CD4 co-receptor. In certain embodiments, the CD4 co-receptor comprises a polypeptide comprising or consisting of an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence having a UniProt Reference No: P01730, or fragments thereof, and/or may optionally comprise up to one or up to two or up to three conservative amino acid substitutions. In certain embodiments, the CD4 co-receptor comprises a polypeptide comprising or consisting of an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 24. In certain embodiments, the CD4 co-receptor comprises a polypeptide comprising or consisting of the amino acid sequence set forth in SEQ ID NO: 24. SEQ ID NO: 24 is provided below:

[SEQ ID NO: 24]
MNRGVPFRHLLLVLQLALLPAATQGKKVVLGKKGDTVELTCTASQKKSIQ
FHWKNSNQIKILGNQGSFLTKGPSKLNDRADSRRSLWDQGNFPLIIKNLK
IEDSDTYICEVEDQKEEVQLLVFGLTANSDTHLLQGQSLTLTLESPPGSS
PSVQCRSPRGKNIQGGKTLSVSQLELQDSGTWTCTVLQNQKKEFKIDIVV
LAFQKASSIVYKKEGEQVEFSFPLAFTVEKLTGSGELWWQAERASSSKSW
ITFDLKNKEVSVKRVTQDPKLQMGKKLPLHLTLPQALPQYAGSGNLTLAL
EAKTGKLHQEVNLVVMRATQLQKNLTCEVWGPTSPKLMLSLKLENKEAKV
SKREKAVWVLNPEAGMWQCLLSDSGQVLLESNIKVLPTWSTPVQPMALIV
LGGVAGLLLFIGLGIFFCVRCRHRRRQAERMSQIKRLLSEKKTCQCPHRF
QKTCSPI

In certain embodiments, cell further comprises at least one recombinant or exogenous co-stimulatory ligand. For example, a presently disclosed cell can be further transduced with at least one co-stimulatory ligand, such that the cell co-expresses or is induced to co-express the presently disclosed TCR and the at least one co-stimulatory ligand. The interaction between the presently disclosed TCR and at least one co-stimulatory ligand provides a non-antigen-specific signal important for full activation of an immunoresponsive cell (e.g., T cell). Co-stimulatory ligands include, but are not limited to, members of the tumor necrosis factor (TNF) superfamily, and immunoglobulin (Ig) superfamily ligands. TNF is a cytokine involved in systemic inflammation and stimulates the acute phase reaction. Its primary role is in the regulation of immune cells. Members of TNF superfamily share a number of common features. The majority of TNF superfamily members are synthesized as type 11 transmembrane proteins (extracellular C-terminus) containing a short cytoplasmic segment and a relatively long extracellular region. TNF superfamily members include, without limitation, nerve growth factor (NGF), CD40L (CD40L)/CD154, CD137L/4-1BBL, TNF-α, CD134L/OX40L/CD252, CD27L/CD70, Fas ligand (FasL), CD30L/CD153, tumor necrosis factor beta (TNF-0)/lymphotoxin-alpha (LTa), lymphotoxin-beta (LTβ), CD257/B cell-activating factor (BAFF)/Blys/THANK/Tall-1, glucocorticoid-induced TNF Receptor ligand (GITRL), and TNF-related apoptosis-inducing ligand (TRAIL), LIGHT (TNFSF14). The immunoglobulin (Ig) superfamily is a large group of cell surface and soluble proteins that are involved in the recognition, binding, or adhesion processes of cells. These proteins share structural features with immunoglobulins—they possess an immunoglobulin domain (fold). Immunoglobulin superfamily ligands include, but are not limited to, CD80 and CD86, both ligands for CD28, PD-L1/(B7-H1) that ligands for PD-1. In certain embodiments, the at least one co-stimulatory ligand is selected from the group consisting of 4-1BBL, CD80, CD86, CD70, OX40L, CD48, TNFRSF14, PD-L1, and combinations thereof. In certain embodiments, the cell comprises one recombinant co-stimulatory ligand that is 4-1BBL. In certain embodiments, the cell comprises two recombinant co-stimulatory ligands that are 4-1BBL and CD80.

In certain embodiments, a presently disclosed cell further comprises at least one exogenous cytokine. For example, a presently disclosed cell can be further transduced with at least one cytokine, such that the cell secretes the at least one cytokine as well as expresses the presently disclosed TCR. In certain embodiments, the at least one cytokine is selected from the group consisting of IL-2, IL-3, IL-6, IL-7, IL-11, IL-12, IL-15, IL-17, IL-18, and IL-21. In certain embodiments, the cytokine is IL-12.

In certain embodiments, a presently disclosed cell further comprises at least one exogenous integrin. For example, a presently disclosed cell can be further transduced with at least one integrin, such that the cell co-expresses or is induced to co-express the presently disclosed TCR and the at least one integrin. In certain embodiments, the at least one integrins is selected from the group consisting of LFA-1 and VLA-4. In certain embodiments, the at least one integrin comprises LFA-1.

### 5.5. Nucleic Acids and Genetic Modifications of Cells

The present discloses subject matter provides a nucleic acid encoding a presently disclosed TCR (e.g., one disclosed in Section 5.3). Further provided are cells comprising such nucleic acids. In certain embodiments, a promoter is operably linked to the presently disclosed TCR.

In certain embodiments, the promoter is endogenous or exogenous. In certain embodiments, the exogenous promoter is selected from the group consisting of a long terminal repeat (LTR) promoter, an elongation factor (EF)-1 promoter, a cytomegalovirus immediate-early promoter (CMV) promoter, a simian virus 40 early promoter (SV40) promoter, a phosphoglycerate kinase (PGK) promoter, and a metallothionein promoter. In certain embodiment, the exogenous promoter is a LTR promoter. In certain embodiments, the promoter is an inducible promoter. In certain embodiment, the inducible promoter is selected from the group consisting of a NFAT transcriptional response element (TRE) promoter, a CD69 promoter, a CD25 promoter, and an IL-2 promoter.

In certain embodiments, the nucleic acid encodes both the α chain and the β chain of a presently disclosed TCR. In certain embodiments, the α chain and the β chain are separated by a self-cleavage peptide, e.g., a 2A-peptide. In certain embodiments, the α chain and the β chain are separated by a furin-2A-peptide. In certain embodiments, the peptide comprises the amino acid sequence set forth in SEQ ID NO: 15. SEQ ID NO: 15 is provided below:
RAKRSGSGATNFSLLKQAGDVEENPGP [SEQ ID NO: 15]

In certain embodiments, the nucleic acid encodes a functional portion/fragment of a presently disclosed TCR. As used herein, the term “functional portion” or “functional fragment” refers to any portion, part or fragment of a presently disclosed TCR, which portion, part or fragment retains the biological activity of the TCR (the parent TCR). For example, functional portions encompass the portions, parts or fragments of a presently disclosed TCR that retains the ability to recognize the RAS peptide (e.g., a RAS peptide comprising a G12D mutation) to a similar, same, or even a higher extent as the parent TCR. In certain embodiments, the nucleic acid encoding a functional portion of a presently disclosed TCR encodes a protein comprising, e.g., about 10%, about 20%, about 25%, about 30%, about 35%, about 40%, about 45%, about 50%, about 55%, about 60%, about 65%, about 70%, about 75%, about 80%, about 85%, about 90%, and about 95%, or more of the parent TCR.

Genetic modification of a cell (e.g., a T cell) can be accomplished by transducing a substantially homogeneous cell composition with a recombinant DNA or RNA construct. In certain embodiments, a retroviral vector (e.g., gamma-retroviral vector or lentiviral vector) is employed for the introduction of the DNA or RNA construct into the cell. For example, a polynucleotide encoding a presently disclosed TCR can be cloned into a retroviral vector and expression can be driven from its endogenous promoter, from the retroviral long terminal repeat, or from an alternative internal promoter, or from a promoter specific for a target cell type of interest. Non-viral vectors or RNA may be used as well. Random chromosomal integration, or targeted integration (e.g., using a nuclease, transcription activator-like effector nucleases (TALENs), Zinc-finger nucleases (ZFNs), and/or clustered regularly interspaced short palindromic repeats (CRISPRs), or transgene expression (e.g., using a natural or chemically modified RNA) can be used. For initial genetic modification of a cell to include a presently disclosed TCR, a retroviral vector can be employed for transduction, however any other suitable viral vector or non-viral delivery system can be used. The TCR can be constructed in a single, multicistronic expression cassette, in multiple expression cassettes of a single vector, or in multiple vectors. Examples of elements that create polycistronic expression cassette include, but is not limited to, various viral and non-viral Internal Ribosome Entry Sites (IRES, e.g., FGF-1 IRES, FGF-2 IRES, VEGF IRES, IGF-II IRES, NF-xB IRES, RUNX1 IRES, p53 IRES, hepatitis A IRES, hepatitis C IRES, pestivirus IRES, aphthovirus IRES, picornavirus IRES, poliovirus IRES and encephalomyocarditis virus IRES) and cleavable linkers (e.g., 2A peptides, e.g., P2A, T2A, E2A and F2A peptides). Combinations of retroviral vector and an appropriate packaging line are also suitable, where the capsid proteins will be functional for infecting human cells. Various amphotropic virus-producing cell lines are known, including, but not limited to, PA12 (Miller et al., (1985) Mol Cell Biol (1985); 5:431-437); PA317 (Miller, et al., Mol Cell Biol (1986); 6:2895-2902); and CRIP (Danos et al., Proc Natl Acad Sci USA (1988); 85:6460-6464). Non-amphotropic particles are suitable too, e.g., particles pseudotyped with VSVG, RD114 or GALV envelope and any other known in the art.

Possible methods of transduction also include direct co-culture of the cells with producer cells (Bregni et al., Blood (1992); 80:1418-1422), or culturing with viral supernatant alone or concentrated vector stocks with or without appropriate growth factors and polycations (Xu et al., Exp Hemat (1994); 22:223-230; and Hughes et al. J Clin Invest (1992); 89:1817).

Other transducing viral vectors can be used to modify a cell. In certain embodiments, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). Other viral vectors that can be used include, for example, adenoviral, lentiviral, and adena-associated viral vectors, vaccinia virus, a bovine papilloma virus, or a herpes virus, such as Epstein-Barr Virus (also see, for example, the vectors of Miller, Human Gene Thera (1990); 15-14; Friedman, Science 244:1275-1281, 1989; Eglitis et al., BioTechniques (1988); 6:608-614; Tolstoshev et al., Cur Opin Biotechnol (1990); 1:55-61; Sharp, The Lancet (1991); 337:1277-78; Cornetta et al., Nucleic Acid Research and Molecular Biology 36:311-22, 1987; Anderson, Science (1984); 226:401-409; Moen, Blood Cells 17:407-16, 1991; Miller et al., Biotechnol (1989); 7:980-90; LeGal La Salle et al., Science (1993); 259:988-90; and Johnson, Chest (1995)107:77S-83S). Retroviral vectors are particularly well developed and have been used in clinical settings (Rosenberg et al., N Engl J Med (1990); 323:370, 1990; Anderson et al., U.S. Pat. No. 5,399,346).

Non-viral approaches can also be employed for genetic modification of a cell. For example, a nucleic acid molecule can be introduced into a cell by administering the nucleic acid in the presence of lipofection (Feigner et al., Proc Natl Acad Sci U.S.A. (1987); 84:7413; Ono et al., Neurosci Lett (1990); 17:259; Brigham et al., Am J Med Sci (1989); 298:278; Staubinger et al., Methods in Enzymol (1983); 101:512, Wu et al., J Biol Chem (1988); 263:14621; Wu et al., J Biol Chem (1989); 264:16985), or by micro-injection under surgical conditions (Wolff et al., Science (1990); 247:1465). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Recombinant receptors can also be derived or obtained using transposases or targeted nucleases (e.g. Zinc finger nucleases, meganucleases, or TALE nucleases, CRISPR). Transient expression may be obtained by RNA electroporation.

In certain embodiments, a presently disclosed TCR can be integrated into a selected locus of the genome of a cell. Any targeted genome editing methods can also be used to deliver a presently disclosed TCR to a cell or a subject. In certain embodiments, a CRISPR system is used to deliver a presently disclosed TCR. In certain embodiments, zinc-finger nucleases are used to deliver presently disclosed TCR. In certain embodiments, a TALEN system is used to deliver a presently disclosed TCR.

In certain embodiments, a presently disclosed TCR can be integrated at a locus encoding a T cell receptor. Non-limiting examples of the loci include a TRAC locus, a TRBC locus, a TRDC locus, and a TRGC locus. In certain embodiments, the locus is a TRAC locus or a TRBC locus. Methods of targeting a TCR to a site within the genome of T cell can be found in WO2017180989 and Eyquem et al., Nature. (2017 Mar. 2); 543(7643): 113-117, both of which are incorporated by reference in their entireties.

In certain embodiments, a presently disclosed TCR can be integrated at a genetic locus encoding an immune-checkpoint. Non-limiting examples of the loci include a PDCD1 locus, a 20 CBLB locus, a CISH locus, or a RASA2 locus. In certain embodiments, the locus is a PDCD1 locus. In certain embodiments, the locus is a CBLB locus. In certain embodiments, the locus is a CISH locus. In certain embodiments, the locus is a RASA2 locus. Non-limiting examples of methods of integrating a presently disclosed TCR to a locus encoding an immune-checkpoint CRISPR systems, zinc-finger nucleases, and TALEN systems.

In certain embodiments, a presently disclosed TCR can be integrated at a genomic safe harbor locus. As used herein, a “genomic safe harbor” or “GSH” refers to a chromosome location where an integrated transgene (e.g., encoding a presently disclosed TCR) can be predictably expressed without adversely affecting endogenous gene structure or expression. In certain embodiments, integrating a transgene at the GSH does not alter cell behavior and/or promote malignant transformation of the host cell or the organism. In certain embodiments, the GSH permits sufficient transgene expression to yield desirable levels of protein or non-coding RNA encoded by the transgene. Additional information on genomic safe harbor sites can be found in International Patent Publication No. WO 2021/055616, Sadelain et al., Nature Reviews Cancer 12.1 (2012): 51-58, and Aznauryan et al., Cell Reports Methods 2.1 (2022), the contents of each of which are incorporated by reference in their entirety.

In certain embodiments, the expression of the TCR is driven by an endogenous promoter/enhancer within or near the locus. In certain embodiments, the expression of the TCR is driven by an exogenous promoter integrated into the locus. The locus where the TCR is integrated is selected based on the expression level of the genes within the locus, and timing of the gene expression of the genes within the locus. The expression level and timing can vary under different stages of cell differentiation and mitogen/cytokine microenvironment, which are among the factors to be considered when making the selection.

In certain embodiments, the CRISPR system is used to integrate the TCR in selected loci of the genome of a cell. In certain embodiments, the CRISPR system uses a DNA donor-template guided homology directed repair at a defined genetic locus, e.g., a TRAC locus. Clustered regularly-interspaced short palindromic repeats (CRISPR) system is a genome editing tool discovered in prokaryotic cells. When utilized for genome editing, the system includes Cas9 (a protein able to modify DNA utilizing crRNA as its guide), CRISPR RNA (crRNA, contains the RNA used by Cas9 to guide it to the correct section of host DNA along with a region that binds to tracrRNA (generally in a hairpin loop form) forming an active complex with Cas9), trans-activating crRNA (tracrRNA, binds to crRNA and forms an active complex with Cas9), and an optional section of DNA repair template (DNA that guides the cellular repair process allowing insertion of a specific DNA sequence). CRISPR/Cas9 often employs a plasmid to transfect the target cells. In certain embodiments, CRISPR/Cas9 is a recombinant ribonucleoprotein complex that is transfected into target cells. The crRNA needs to be designed for each application as this is the sequence that Cas9 uses to identify and directly bind to the target DNA in a cell. The repair template carrying TCR expression cassette need also be designed for each application, as it must overlap with the sequences on either side of the cut and code for the insertion sequence. Multiple crRNA's and the tracrRNA can be packaged together to form a single-guide RNA (sgRNA). This sgRNA can be joined together with the Cas9 gene and made into a plasmid in order to be transfected into cells. Methods of using the CRISPR system are described, for example, in WO 2014093661 A2, WO 2015123339 A1 and WO 2015089354 A1, which are incorporated by reference in their entireties.

In certain embodiments, zinc-finger nucleases are used to integrate the TCR in selected loci of the genome of a cell. A zinc-finger nuclease (ZFN) is an artificial restriction enzyme, which is generated by combining a zinc finger DNA-binding domain with a DNA-cleavage domain. A zinc finger domain can be engineered to target specific DNA sequences which allows a zinc-finger nuclease to target desired sequences within genomes. The DNA-binding domains of individual ZFNs typically contain a plurality of individual zinc finger repeats and can each recognize a plurality of base pairs. The most common method to generate new zinc-finger domain is to combine smaller zinc-finger “modules” of known specificity. The most common cleavage domain in ZFNs is the non-specific cleavage domain from the type IIs restriction endonuclease FokI. Using the endogenous homologous recombination (HR) machinery and a homologous DNA template carrying TCR expression cassette, ZFNs can be used to insert the TCR expression cassette into genome. When the targeted sequence is cleaved by ZFNs, the HR machinery searches for homology between the damaged chromosome and the homologous DNA template, and then copies the sequence of the template between the two broken ends of the chromosome, whereby the homologous DNA template is integrated into the genome. Methods of using the ZFN system are described, for example, in WO 2009146179 A1, WO 2008060510 A2 and CN 102174576 A, which are incorporated by reference in their entireties.

In certain embodiments, the TALEN system is used to integrate the TCR in selected loci of the genome of an immunoresponsive cell. Transcription activator-like effector nucleases (TALEN) are restriction enzymes that can be engineered to cut specific sequences of DNA. TALEN system operates on almost the same principle as ZFNs. They are generated by combining a transcription activator-like effectors DNA-binding domain with a DNA cleavage domain. Transcription activator-like effectors (TALEs) are composed of 33-34 amino acid repeating motifs with two variable positions that have a strong recognition for specific nucleotides. By assembling arrays of these TALEs, the TALE DNA-binding domain can be engineered to bind desired DNA sequence, and thereby guide the nuclease to cut at specific locations in genome. Methods of using the TALEN system are described, for example, in WO 2014134412 A1, WO 2013163628 A2 and WO 2014040370 A1, which are incorporated by reference in their entireties.

cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element or intron (e.g. the elongation factor 1a enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

Methods for delivering the genome editing agents/systems can vary depending on the need. In certain embodiments, the components of a selected genome editing method are delivered as DNA constructs in one or more plasmids. In certain embodiments, the components are delivered via viral vectors. Common delivery methods include but is not limited to, electroporation, microinjection, gene gun, impalefection, hydrostatic pressure, continuous infusion, sonication, magnetofection, adeno-associated viruses, envelope protein pseudotyping of viral vectors, replication-competent vectors cis and trans-acting elements, herpes simplex virus, and chemical vehicles (e.g., oligonucleotides, lipoplexes, polymersomes, polyplexes, dendrimers, inorganic Nanoparticles, and cell-penetrating peptides).

In certain embodiments, the delivery methods include the use of colloids. As used herein, the term “colloid” refers to systems in which there are two or more phases, with one phase (e.g., the dispersed phase) distributed in the other phase (e.g., the continuous phase). Moreover, at least one of the phases has small dimensions (in the range of about 10-9 to about 10-6 m). Non-limiting examples of colloids encompassed by the presently disclosed subject matter include macromolecule complexes, nanocapsules, microspheres, beads, and lipid-based systems (e.g., micelles, liposomes, and lipid nanoparticles).

In certain embodiments, the delivery methods include the use of liposomes. The term “liposome,” as used herein, refers to single- or multi-layered spherical lipid bilayer structures produced from lipids dissolved in organic solvents and then dispersed in aqueous media. Experimentally and therapeutically used for delivering an active pharmaceutical ingredient (e.g., nucleic acid compositions disclosed herein) to cells, liposomes fuse with cell membranes so the contents are transferred into the cytoplasm.

In certain embodiments, the delivery methods include the use of lipid nanoparticles. As used herein, the term “lipid nanoparticle” refers to a particle having at least one dimension in the order of nanometers (e.g., from about 1 nm to about 1,000 nm) and including at least one lipid. In certain embodiments, the lipid nanoparticles can include an active pharmaceutical ingredient (e.g., nucleic acid compositions disclosed herein) for delivering to cells. The morphology of the lipid nanoparticles can be different from liposomes. While liposomes are characterized by a lipid bilayer surrounding a hydrophilic core, lipid nanoparticles have an electron-dense core where cationic lipids and/or ionizable lipids are organized into inverted micelles around an active pharmaceutical ingredient (e.g., nucleic acid compositions disclosed herein). Additional information on the morphology and properties of lipid nanoparticles and liposomes can be found in Wilczewska, et al., Pharmacological reports 64, no. 5 (2012): 1020-1037; Eygeris et al., Accounts of Chemical Research 55, no. 1 (2021): 2-12; Zhang et al., Chemical Reviews 121, no. 20 (2021): 12181-12277; and Fan et al., Journal of pharmaceutical and biomedical analysis 192 (2021): 113642.

In certain embodiments, the lipid nanoparticles have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm.

In certain embodiments, the lipid nanoparticles can include a cationic lipid or an ionizable lipid. The term “cationic lipid” refers to lipids including a head group with permanent positive charges. Non-limiting examples of cationic lipids encompassed by the presently disclosed subject matter include 1,2-di-O-octadecenyl-3-trimethylammonium-propane (DOTMA), 1,2-dioleoyl-3-trimethylammonium-propane (DOTAP), 2,3-dioleyloxy-N-[2-(sperminecarboxamido)ethyl]-N,N-dimethyl-1-propanaminium trifluoroacetate (DOSPA), and ethylphosphatidylcholine (ePC).

As used herein, the term “ionizable lipid” refers to lipids that are protonated at low pH and are neutral at physiological pH. The pH-sensitivity of ionizable lipids is particularly beneficial for delivery in vivo (e.g., delivery of nucleic acid compositions disclosed herein), because neutral lipids have less interactions with the anionic membranes of blood cells and, thus, improve the biocompatibility of the lipid nanoparticles. Once trapped in endosomes, ionizable lipids are protonated and promote membrane destabilization to allow the endosomal escape of the nanoparticles. Non-limiting example of ionizable lipids encompassed by the presently disclosed subject matter include tetrakis(8-methylnonyl) 3,3′,3″,3′″-(((methylazanediyl) bis(propane-3,1 diyl))bis (azanetriyl))tetrapropionate; decyl (2-(dioctylammonio)ethyl) phosphate; ((4-hydroxybutyl)azanediyl)bis(hexane-6,1-diyl)bis(2-hexyldecanoate); bis(2-(dodecyldisulfanyl)ethyl) 3,3′-((3-methyl-9-oxo-10-oxa-13,14-dithia-3,6-diazahexacosyl)azanediyl)dipropionate; 1,1′-((2-(4-(2-((2-(bis(2-hydroxydodecyl)amino)ethyl) (2-hydroxydodecyl)amino)ethyl) piperazin-1-yl)ethyl)azanediyl) bis(dodecan-2-ol); cKK-E12, 3,6-bis(4-(bis(2-hydroxydodecyl)amino)butyl)piperazine-2,5-dione; (6Z,9Z,28Z,31Z)-heptatriaconta-6,9,28,31-tetraen-19-yl 4-(dimethylamino) butanoate; hexa(octan-3-yl) 9,9′,9″,9′″,9″″,9′″″-((((benzene-1,3,5-tricarbonyl)yris(azanediyl)) tris (propane-3,1-diyl)) tris(azanetriyl))hexanonanoate; heptadecan-9-yl 8-((2-hydroxyethyl)(6-oxo-6-(undecyloxy)hexyl)amino) octanoate; and (((3,6-dioxopiperazine-2,5-diyl)bis(butane-4, 1-diyl))bis(azanetriyl))tetrakis(ethane-2,1-diyl) (9Z,9′ Z,9″ Z,9′″Z,12Z,12′ Z,12″ Z,12′″Z)-tetrakis (octadeca-9,12-dienoate).

Additionally, in certain embodiments, the lipid nanoparticles can include other lipids. For example, but without any limitation, the lipid nanoparticles of the presently disclosed subject matter can include phospholipids, cholesterol, polyethylene glycol (PEG)-functionalized lipids (PEG-lipids). These lipids can improve certain properties of the lipid nanoparticles (e.g., stability, biodistribution, etc.). For example, cholesterol enhances the stability of the lipid nanoparticles by modulating their integrity and rigidity. Non-limiting examples of other lipids present in lipid nanoparticles include cholesterol, DC-cholesterol, 0-sitosterol, BHEM-cholesterol, ALC-0159, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoylphosphatidylethanolamine (DSPE), 16-O-monomethyl PE, 16-O-dimethyl PE, 18-1-trans PE, 1-stearioyl-2-oleoyl-phosphatidyethanol amine (SOPE), and 1,2-dielaidoyl-sn-glycero-3-phophoethanolamine (transDOPE).

In certain embodiments, the lipid nanoparticles can include a targeting moiety that binds to a ligand. The use of the targeting moieties allows selective delivery of an active pharmaceutical ingredient (e.g., nucleic acid compositions disclosed herein) to target cells expressing the ligand (e.g., T cells). In certain embodiments, the targeting moiety can be an antibody or antigen-binding fragment thereof that binds to a cell surface receptor. For example, but without any limitation, the targeting domain is an antibody or antigen-binding fragment thereof that binds to a receptor expressed on the surface of a T cell (e.g., CD3, CD4, CD8, CD16, CD40L, CD95, FasL, CTLA-4, OX40, GITR, LAG3, ICOS, and PD-1).

In certain embodiments, the delivery methods are in vivo delivery methods. In certain embodiments, the delivery methods are ex vivo delivery methods.

Modification can be made anywhere within the selected locus, or anywhere that can influence gene expression of the integrated TCR. In certain embodiments, the modification is introduced upstream of the transcriptional start site of the integrated TCR. In certain embodiments, the modification is introduced between the transcriptional start site and the protein coding region of the integrated TCR. In certain embodiments, the modification is introduced downstream of the protein coding region of the integrated TCR.

### 5.6. Formulations and Administration

The presently disclosed subject matter also provides compositions comprising the presently disclosed cells (e.g., those disclosed in Section 5.4). In certain embodiments, the composition is a pharmaceutical composition that further comprises a pharmaceutically acceptable carrier.

Compositions comprising the presently disclosed cells can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Compositions comprising the presently disclosed cells can be provided systemically or directly to a subject for inducing and/or enhancing an immune response to an antigen and/or treating and/or preventing a tumor. In certain embodiments, the presently disclosed cells or compositions comprising thereof are directly injected into an organ of interest (e.g., an organ affected by a neoplasm). Alternatively, the presently disclosed cells or compositions comprising thereof are provided indirectly to the organ of interest, for example, by administration into the circulatory system (e.g., the tumor vasculature). Expansion and differentiation agents can be provided prior to, during or after administration of the cells or compositions to increase production of cells in vitro or in vivo.

The quantity of cells to be administered can vary for the subject being treated. In certain embodiments, between about 10⁴ and about 10¹¹, between about 10⁴ and about 10⁷, between about 10⁵ and about 10⁷, between about 10⁵ and about 10⁹, or between about 10⁶ and about 10⁸ of the presently disclosed cells are administered to a subject. In certain embodiments, at least about 1×10⁵ cells can be administered, eventually reaching about 1×10¹⁰ or more. In certain embodiments, at least about 1×10⁶ cells can be administered. In certain embodiments, from about 10⁴ to about 10¹¹, from about 10⁵ to about 10⁹, or from about 10⁶ to about 10⁸ the presently disclosed cells are administered to a subject. More effective cells may be administered in even smaller numbers. In certain embodiments, at least about 1×10⁸, about 2×10⁸, about 3×10⁸, about 4×10⁸, and about 5×10⁸ the presently disclosed cells are administered to a subject. The precise determination of what would be considered an effective dose can be based on factors individual to each subject, including their size, age, sex, weight, and condition of the particular subject. Dosages can be readily ascertained by those skilled in the art from this disclosure and the knowledge in the art.

The presently disclosed cells and compositions can be administered by any method known in the art including, but not limited to, intravenous administration, subcutaneous administration, intranodal administration, intratumoral administration, intrathecal administration, intrapleural administration, intraosseous administration, intraperitoneal administration, pleural administration, and direct administration to the subject. The presently disclosed cells can be administered in any physiologically acceptable vehicle, normally intravascularly, although they may also be introduced into bone or other convenient site where the cells may find an appropriate site for regeneration and differentiation (e.g., thymus).

### 5.7. Methods of Treatment

The presently disclosed subject matter provides various methods of using the presently disclosed cells or compositions comprising thereof. The presently disclosed cells and compositions comprising thereof can be used in a therapy or medicament. For example, the presently disclosed subject matter provides methods for inducing and/or increasing an immune response in a subject in need thereof. The presently disclosed cells and compositions comprising thereof can be used for reducing tumor burden in a subject. The presently disclosed cells and compositions comprising thereof can reduce the number of tumor cells, reduce tumor size, and/or eradicate the tumor in the subject. The presently disclosed cells and compositions comprising thereof can be used for treating and/or preventing a tumor in a subject. The presently disclosed cells and compositions comprising thereof can be used for prolonging the survival of a subject suffering from a tumor.

In certain embodiments, each of the above-noted methods comprises administering the presently disclosed cells or a composition (e.g., a pharmaceutical composition) comprising thereof to achieve the desired effect, e.g., palliation of an existing condition or prevention of recurrence of tumor. For treatment, the amount administered is an amount effective in producing the desired effect. An effective amount can be provided in one or a series of administrations. An effective amount can be provided in a bolus or by continuous perfusion.

In certain embodiments, the tumor is associated with RAS. In certain embodiments, the tumor is associated with a RAS mutation or a RAS mutant. In certain embodiments, the RAS mutation is a G12 mutation. In certain embodiments, the RAS mutation is a G12D mutation.

In certain embodiments, the tumor is a cancer. In certain embodiments, the tumor is selected from the group consisting of pancreatic cancer, breast cancer, endometrial cancer, cervical cancer, anal cancer, bladder cancer, colorectal cancer, cholangiocarcinoma/bile duct cancer, lung cancer, ovarian cancer, esophageal cancer, gastric cancer (also known as “stomach cancer”), head and neck squamous cell carcinoma, nonmelanoma skin cancer, salivary gland cancer, melanoma, and multiple myeloma. In certain embodiments, the cancer is colorectal cancer.

Additionally, the presently disclosed cells and compositions comprising thereof can be used for treating and/or preventing a premalignant condition in a subject. The presently disclosed cells and compositions comprising thereof can be used for prolonging the survival of a subject suffering from a premalignant condition. As used herein, the term “premalignant condition” refers to any condition that, if left untreated, can turn into cancer. In certain embodiments, the premalignant condition can be characterized by a precancerous lesion that is a morphologically altered tissue in which cancer is more likely to occur than in its apparently normal counterpart. In certain embodiments the premalignant condition is clonal hematopoiesis. In certain embodiments the premalignant condition is myelodysplastic syndrome.

In certain embodiments, the subject is a human subject. The subjects can have an advanced form of disease, in which case the treatment objective can include mitigation or reversal of disease progression, and/or amelioration of side effects. The subjects can have a history of the condition, for which they have already been treated, in which case the therapeutic objective will typically include a decrease or delay in the risk of recurrence.

In certain embodiments, the subject comprises an HLA-A. In certain embodiments, the HLA-A is an HLA-A*03 superfamily member. In certain embodiments, the HLA-A*03 superfamily member is selected from the group consisting of HLA-A*03, HLA-A*11, HLA-A*31, HLA-A*33, HLA-A*66, HLA-A*68 and HLA-A*74. In certain embodiments, the HLA-A*03 superfamily member is HLA-A*11.

### 5.8. Diagnostic and Prognostic Methods

The presently disclosed TCR, multi-specific molecules, and nucleic acids encode thereof can be used for diagnostic and prognostic applications as well as use as research tools for detection of RAS in a biological sample, in a cell, a tissue, or a blood sample. The presently disclosed subject matter provides methods for detecting RAS in a cell, a tissue, or a blood sample. In certain embodiments, the method comprises: contacting a cell, a tissue, or a blood sample with presently disclosed TCR (or a functional fragment thereof) or multi-specific molecule disclosed herein, wherein the TCR or multi-specific molecule comprises a detectable label; and determining the amount of the labeled TCR or multi-specific molecule bound to the cell, tissue, or blood sample by measuring the amount of detectable label associated with the cell or tissue, wherein the amount of bound TCR or multi-specific molecule indicates the amount of RAS in the cell, tissue, or a blood sample. The cell or tissue can be any cell or tissue, including any normal, healthy, or cancerous cells and tissues. In certain embodiments, the blood sample is a peripheral blood sample.

The presently disclosed TCR (or a functional fragment thereof) can be used in methods known in the art relating to the localization and/or quantitation of RAS polypeptides (e.g., for use in measuring levels of the RAS protein within appropriate physiological samples, for use in diagnostic methods, for use in imaging the polypeptide, and the like). The presently disclosed TCR (or a functional fragment thereof) can be used to isolate a cell including a RAS polypeptide by standard techniques, such as affinity chromatography or immunoprecipitation. Moreover, the presently disclosed TCR (or a functional fragment thereof) can be used to detect an immunoreactive RAS protein (e.g., in plasma, a cellular lysate or cell supernatant) in order to evaluate the abundance and pattern of expression of the immunoreactive polypeptide. the presently disclosed TCR (or a functional fragment thereof) can be used diagnostically to monitor immunoreactive RAS protein levels in tissue as part of a clinical testing procedure, e.g., to determine the efficacy of a given treatment regimen. As noted above, the detection can be facilitated by coupling (i.e., physically linking) the presently disclosed TCR (or a functional fragment thereof) to a detectable substance.

An exemplary method for detecting the presence or absence of an immunoreactive RAS protein in a biological sample comprises contacting a biological sample from a subject with the presently disclosed TCR (or a functional fragment thereof), wherein the presence of an immunoreactive RAS protein is detected in the biological sample. Detection may be accomplished by means of a detectable label attached to the antibody.

The term “labeled” with regard to the presently disclosed TCR (or a functional fragment thereof) is intended to encompass direct labeling of the TCR by coupling (i.e., physically linking) a detectable substance to the antibody, as well as indirect labeling of the antibody by reactivity with another compound that is directly labeled, such as a secondary antibody.

In certain embodiments, the presently disclosed TCR (or a functional fragment thereof) is conjugated to one or more detectable labels. For such uses, the presently disclosed TCR (or a functional fragment thereof) may be detectably labeled by covalent or non-covalent attachment of a chromogenic, enzymatic, radioisotopic, isotopic, fluorescent, toxic, chemiluminescent, nuclear magnetic resonance contrast agent or other label.

The presently disclosed detection methods can be used to detect an immunoreactive RAS protein in a biological sample in vitro as well as in vivo. Non-limiting examples of in vitro techniques for detection of an immunoreactive RAS protein include enzyme linked immunosorbent assays (ELISAs), Western blots, immunoprecipitations, radioimmunoassay, and immunofluorescence. Furthermore, in vivo techniques for detection of an immunoreactive RAS protein include introducing into a subject a labeled TCR (or a functional fragment thereof). For example, the presently disclosed TCR (or a functional fragment thereof) can be labeled with a radioactive marker whose presence and location in a subject can be detected by standard imaging techniques. In certain embodiments, the biological sample comprises RAS protein molecules from the test subject.

The presently disclosed TCR (or a functional fragment thereof) can be used to assay immunoreactive RAS protein levels in a biological sample (e.g., human plasma) using antibody-based techniques. For example, protein expression in tissues can be studied with classical immunohistological methods. Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase, and radioisotopes or other radioactive agent, such as iodine (125I, 121I, 131I), carbon (14C), sulfur (35S), tritium (3H), indium (111In), and technetium (99mTc), and fluorescent labels, such as fluorescein, rhodamine, and green fluorescent protein (GFP), as well as biotin.

In addition to assaying immunoreactive RAS protein levels in a biological sample, the presently disclosed TCR (or a functional fragment thereof) may be used for in vivo imaging of RAS. Antibodies useful for this method include those detectable by X-radiography, NMR or ESR. For X-radiography, suitable labels include radioisotopes such as barium or cesium, which emit detectable radiation but are not overtly harmful to the subject. Suitable markers for NMR and ESR include those with a detectable characteristic spin, such as deuterium, which can be incorporated into the presently disclosed TCR (or a functional fragment thereof).

The presently disclosed TCR (or a functional fragment thereof), which are labeled with an appropriate detectable imaging moiety (such as a radioisotope (e.g., ¹³¹, ¹¹¹In, ⁹⁹mTc, ¹⁸F, ⁸⁹Zr), a radio-opaque substance, or a material detectable by nuclear magnetic resonance) are introduced (e.g., parenterally, subcutaneously, or intraperitoneally) into the subject. It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries. The labeled TCR (or a functional fragment thereof) then accumulates at the location of cells which contain the specific target polypeptide. For example, the presently disclosed TCR (or a functional fragment thereof) accumulate within the subject in cells and tissues in which the RAS protein has localized.

Thus, the presently disclosed subject matter provides diagnostic methods of a medical condition. In certain embodiments, the method comprises: (a) assaying the expression of immunoreactive RAS protein by measuring binding of a presently disclosed TCR (or a functional fragment thereof) in cells or body fluid of an individual; and (b) comparing the amount of immunoreactive RAS protein present in the sample with a standard reference, wherein an increase or decrease in immunoreactive RAS protein levels compared to the standard is indicative of a medical condition.

Furthermore, the presently disclosed TCR (or a functional fragment thereof) may be used to purify cells including a RAS protein from a sample. In certain embodiments, the TCRs are immobilized on a solid support. Non-limiting examples of such solid supports include plastics such as polycarbonate, complex carbohydrates such as agarose and sepharose, acrylic resins and such as polyacrylamide and latex beads. Techniques for coupling TCRs to such solid supports are well known in the art.

A TCR or polypeptide of interest can be conjugated to a solid support, such as a bead. In addition, a first solid support such as a bead can also be conjugated, if desired, to a second solid support, which can be a second bead or other support, by any suitable means, including those disclosed herein for conjugation of a polypeptide to a support. Accordingly, any of the conjugation methods and means disclosed herein with reference to conjugation of a polypeptide to a solid support can also be applied for conjugation of a first support to a second support, where the first and second solid support can be the same or different.

Appropriate linkers, which can be cross-linking agents, for use for conjugating a polypeptide to a solid support include a variety of agents that can react with a functional group present on a surface of the support, or with the polypeptide, or both. Reagents useful as cross-linking agents include homo-bi-functional and, in particular, hetero-bi-functional reagents. Useful bi-functional cross-linking agents include, but are not limited to, N-SIAB, dimaleimide, DTNB, N-SATA, N-SPDP, SMCC and 6-HYNIC. A cross-linking agent can be selected to provide a selectively cleavable bond between a polypeptide and the solid support. For example, a photolabile cross-linker, such as 3-amino-(2-nitrophenyl)propionic acid can be employed as a means for cleaving a polypeptide from a solid support. (Brown et al., Mol. Divers, pp, 4-12 (1995); Rothschild et al., Nucl. Acids Res., 24:351-66 (1996); and U.S. Pat. No. 5,643,722). Other cross-linking reagents are well-known in the art. (See, e.g., Wong (1991), supra; and Hermanson (1996), supra).

A TCR or polypeptide can be immobilized on a solid support, such as a bead, through a covalent amide bond formed between a carboxyl group functionalized bead and the amino terminus of the polypeptide or, conversely, through a covalent amide bond formed between an amino group functionalized bead and the carboxyl terminus of the polypeptide. In addition, a bi-functional trityl linker can be attached to the support, e.g., to the 4-nitrophenyl active ester on a resin, such as a Wang resin, through an amino group or a carboxyl group on the resin via an amino resin. Using a bi-functional trityl approach, the solid support can require treatment with a volatile acid, such as formic acid or trifluoroacetic acid to ensure that the polypeptide is cleaved and can be removed. In such a case, the polypeptide can be deposited as a beadless patch at the bottom of a well of a solid support or on the flat surface of a solid support. After addition of a matrix solution, the polypeptide can be desorbed into a MS.

Hydrophobic trityl linkers can also be exploited as acid-labile linkers by using a volatile acid or an appropriate matrix solution, e.g., a matrix solution containing 3-HPA, to cleave an amino linked trityl group from the polypeptide. Acid lability can also be changed. For example, trityl, monomethoxytrityl, dimethoxytrityl or trimethoxytrityl can be changed to the appropriate p-substituted, or more acid-labile tritylamine derivatives, of the polypeptide, i.e., trityl ether and tritylamine bonds can be made to the polypeptide. Accordingly, a polypeptide can be removed from a hydrophobic linker, e.g., by disrupting the hydrophobic attraction or by cleaving tritylether or tritylamine bonds under acidic conditions, including, if desired, under typical MS conditions, where a matrix, such as 3-HPA acts as an acid.

Orthogonally cleavable linkers can also be useful for binding a first solid support, e.g., a bead to a second solid support, or for binding a polypeptide of interest to a solid support. Using such linkers, a first solid support, e.g., a bead, can be selectively cleaved from a second solid support, without cleaving the polypeptide from the support; the polypeptide then can be cleaved from the bead at a later time. For example, a disulfide linker, which can be cleaved using a reducing agent, such as DTT, can be employed to bind a bead to a second solid support, and an acid cleavable bi-functional trityl group could be used to immobilize a polypeptide to the support. As desired, the linkage of the polypeptide to the solid support can be cleaved first, e.g., leaving the linkage between the first and second support intact. Trityl linkers can provide a covalent or hydrophobic conjugation and, regardless of the nature of the conjugation, the trityl group is readily cleaved in acidic conditions.

For example, a bead can be bound to a second support through a linking group which can be selected to have a length and a chemical nature such that high density binding of the beads to the solid support, or high density binding of the polypeptides to the beads, is promoted. Such a linking group can have, e.g., “tree-like” structure, thereby providing a multiplicity of functional groups per attachment site on a solid support. Examples of such linking group; include polylysine, polyglutamic acid, penta-erythrole and tris-hydroxy-aminomethane.

Noncovalent Binding Association. A TCR or polypeptide can be conjugated to a solid support, or a first solid support can also be conjugated to a second solid support, through a noncovalent interaction. For example, a magnetic bead made of a ferromagnetic material, which is capable of being magnetized, can be attracted to a magnetic solid support, and can be released from the support by removal of the magnetic field. Alternatively, the solid support can be provided with an ionic or hydrophobic moiety, which can allow the interaction of an ionic or hydrophobic moiety, respectively, with a polypeptide, e.g., a polypeptide containing an attached trityl group or with a second solid support having hydrophobic character.

A solid support can also be provided with a member of a specific binding pair and, therefore, can be conjugated to a polypeptide or a second solid support containing a complementary binding moiety. For example, a bead coated with avidin or with streptavidin can be bound to a polypeptide having a biotin moiety incorporated therein, or to a second solid support coated with biotin or derivative of biotin, such as iminobiotin.

It should be recognized that any of the binding members disclosed herein or otherwise known in the art can be reversed. Thus, biotin, e.g., can be incorporated into either a polypeptide or a solid support and, conversely, avidin or other biotin binding moiety would be incorporated into the support or the polypeptide, respectively. Other specific binding pairs contemplated for use herein include, but are not limited to, hormones and their receptors, enzyme, and their substrates, a nucleotide sequence and its complementary sequence, an antibody and the antigen to which it interacts specifically, and other such pairs knows to those skilled in the art.

The presently disclosed TCR (or a functional fragment thereof) is useful in diagnostic methods. As such, the presently disclosed subject matter provides methods using the presently disclosed TCR (or a functional fragment thereof) in diagnosis of RAS activity in a subject. The presently disclosed TCR (or a functional fragment thereof) may be selected such that they have any level of epitope binding specificity and high binding affinity to a RAS polypeptide.

The presently disclosed TCR (or a functional fragment thereof) can be used to detect an immunoreactive RAS protein in a variety of standard assay formats. Such formats include immunoprecipitation, Western blotting, ELISA, radioimmunoassay, and immunometric assays.

Biological samples can be obtained from any tissue or body fluid of a subject. In certain embodiments, the subject is at an early stage of cancer. In certain embodiments, the early stage of cancer is determined by the level or expression pattern of RAS protein in a sample obtained from the subject. In certain embodiments, the sample is selected from the group consisting of urine, blood, serum, plasma, saliva, amniotic fluid, cerebrospinal fluid (CSF), and biopsied body tissue.

In certain embodiments, the presently disclosed TCR (or a functional fragment thereof) is conjugated to a diagnostic agent. The diagnostic agent may comprise a radioactive or non-radioactive label, a contrast agent (such as for magnetic resonance imaging, computed tomography or ultrasound), and the radioactive label can be a gamma-, beta-, alpha-, Auger electron-, or positron-emitting isotope. A diagnostic agent is a molecule which is administered conjugated to an antibody moiety, i.e., antibody or antibody fragment, or subfragment, and is useful in diagnosing or detecting a disease by locating the cells comprising the antigen.

Useful diagnostic agents include, but are not limited to, radioisotopes, dyes (such as with the biotin-streptavidin complex), contrast agents, fluorescent compounds or molecules and enhancing agents (e.g., paramagnetic ions) for magnetic resonance imaging (MRI). In certain embodiments, the diagnostic agents are selected from the group consisting of radioisotopes, enhancing agents for use in magnetic resonance imaging, and fluorescent compounds. Chelates may be coupled to the presently disclosed TCR (or a functional fragment thereof) using standard chemistries. The chelate is normally linked to the antibody by a group which enables formation of a bond to the molecule with minimal loss of immunoreactivity and minimal aggregation and/or internal cross-linking.

### 5.9. Kits

The presently disclosed subject matter provides kits for treatment or ameliorating a disease or disorder associated with RAS (e.g., a metastatic cancer cell), and/or detecting RAS. In certain embodiments, the kit comprises the presently disclosed TCR (or a functional fragment thereof), the cells, the multi-specific molecule, or the composition disclosed herein. In certain embodiments, the kit comprises a sterile container which contains a therapeutic or prophylactic vaccine; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

In certain embodiments, the kit further comprises instructions for administering the presently disclosed TCR (or a functional fragment thereof), the cells, the multi-specific molecule, or the composition disclosed herein to a subject in need the treatment. The instructions can generally include information about the use of the presently disclosed TCR (or a functional fragment thereof), the cells, the multi-specific molecule, and the composition disclosed herein for the treatment or ameliorating a disease or disorder. In certain embodiments, the instructions include at least one of the following: description of the therapeutic agent; dosage schedule and administration for treatment and/or prevention of a tumor or neoplasm or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

### 5.10. Exemplary Embodiments

A1. In certain non-limiting embodiments, the presently disclosed subject matter provides a T cell receptor (TCR) that binds to a RAS peptide, wherein the RAS peptide comprises a G12 mutation.
A2. The foregoing TCR of A1, wherein the RAS peptide comprises a G12D mutation.
A3. The foregoing TCR of A1 or A2, wherein the RAS peptide is 9-mer or 10-mer.
A4. The foregoing TCR of any one of A1-A3, wherein the RAS peptide comprises or consists of the amino acid sequence set forth in SEQ ID NO: 1, SEQ ID NO: 2.
A5. The foregoing TCR of any one of A1-A4, wherein the RAS peptide comprises or consists of the amino acid sequence set forth in SEQ ID NO: 2.
A6. The foregoing TCR of A1, wherein the RAS peptide comprises a G12V mutation.
A7. The foregoing TCR of A1 or A6, wherein the RAS peptide is 9-mer or 10-mer.
A8. The foregoing TCR of any one of A1, A6, or A7, wherein the RAS peptide comprises or consists of the amino acid sequence set forth in SEQ ID NO: 4 or SEQ ID NO: 5.
A9. The foregoing TCR of A1, wherein the RAS peptide comprises a G12C mutation.
A10. The foregoing TCR of A1 or A9, wherein the RAS peptide is 10-mer.
All. The foregoing TCR of any one of A1, A9, or A10, wherein the RAS peptide comprises or consists of the amino acid sequence set forth in SEQ ID NO: 6.
A12. The foregoing TCR of any one of A1-A11, wherein the RAS peptide is associated with an HLA class I complex.
A13. The foregoing TCR of A12, wherein the HLA class I complex is selected from an HLA-A, an HLA-B, and an HLA-C.
A14. The foregoing TCR of A12 or A13, wherein the HLA class I complex is an HLA-A.
A15. The foregoing TCR of A13 or A14, wherein the HLA-A is an HLA-A*03 superfamily member.
A16. The foregoing TCR of A15, wherein the HLA-A*03 superfamily member is selected from the group consisting of HLA-A*03, HLA-A*11, HLA-A*31, HLA-A*33, HLA-A*66, HLA-A*68 and HLA-A*74.
A17. The foregoing TCR of A15 or A16, wherein the HLA-A*03 superfamily member is HLA-A*11.
A18. The foregoing TCR of any one of A1-A17, wherein the TCR comprises an extracellular domain that binds to the RAS peptide, wherein the extracellular domain comprises an α chain and a β chain, wherein the α chain comprises an α chain variable region and α chain constant region, and the β chain comprises a β chain variable region and a β chain constant region.
A19. The foregoing TCR of A18, wherein the α chain variable region comprises a CDR3 comprising the amino acid sequence set forth in SEQ ID NO: 18 or a conservative modification thereof, and the β chain variable region comprises a CDR3 comprising the amino acid sequence set forth in SEQ ID NO: 21 or a conservative modification thereof.
A20. The foregoing TCR of A18 or A19, wherein the α chain variable region comprises a CDR2 comprising the amino acid sequence set forth in SEQ ID NO: 17 or a conservative modification thereof, and the β chain variable region comprises a CDR2 comprising the amino acid sequence set forth in SEQ ID NO: 20 or a conservative modification thereof.
A21. The foregoing TCR of any one of A18-A20, wherein the α chain variable region comprises a CDR1 comprising the amino acid sequence set forth in SEQ ID NO: 16 or a conservative modification thereof, and the β chain variable region comprises a CDR1 comprising the amino acid sequence set forth in SEQ ID NO: 19 or a conservative modification thereof.
A22. The foregoing TCR of any one of A18-A21, wherein the α chain variable region comprises a CDR1 comprising the amino acid sequence set forth in SEQ ID NO: 16 or a conservative modification thereof, a CDR2 comprising the amino acid sequence set forth in SEQ ID NO: 17 or a conservative modification thereof, and a CDR3 comprising the amino acid sequence set forth in SEQ ID NO: 18 or a conservative modification thereof, and the β chain variable region comprises a CDR1 comprising the amino acid sequence set forth in SEQ ID NO: 19 or a conservative modification thereof, a CDR2 comprising the amino acid sequence set forth in SEQ ID NO: 20 or a conservative modification thereof, and a CDR3 comprising the amino acid sequence set forth in SEQ ID NO: 21 or a conservative modification thereof.
A23. The foregoing TCR of any one of A18-A22, wherein the α chain variable region comprises a CDR1 comprising the amino acid sequence set forth in SEQ ID NO: 16, a CDR2 comprising the amino acid sequence set forth in SEQ ID NO: 17, and a CDR3 comprising the amino acid sequence set forth in SEQ ID NO: 18; and the β chain variable region comprises a CDR1 comprising the amino acid sequence set forth in SEQ ID NO: 19, a CDR2 comprising the amino acid sequence set forth in SEQ ID NO: 20, and a CDR3 comprising the amino acid sequence set forth in SEQ ID NO: 21.
A24. The foregoing TCR of any one of A18-A23, wherein the α chain variable region comprises an amino acid sequence that is at least about 80% homologous or identical to the amino acid sequence set forth in SEQ ID NO: 7.
A25. The foregoing TCR of A24, wherein the α chain variable region comprises the amino acid sequence set forth in SEQ ID NO: 7.
A26. The foregoing TCR of any one of A18-A25, wherein the β chain variable region comprises an amino acid sequence that is at least about 80% homologous or identical to the amino acid sequence set forth in SEQ ID NO: 8.
A27. The foregoing TCR of A26, wherein the β chain variable region comprises the amino acid sequence set forth in SEQ ID NO: 8.
A28. The foregoing TCR of any one of A18-A27, wherein the α chain variable region comprises an amino acid sequence that is at least about 80% homologous or identical to the amino acid sequence set forth in SEQ ID NO: 7, and the β chain variable region comprises an amino acid sequence that is at least about 80% homologous or identical to the amino acid sequence set forth in SEQ ID NO: 8.
A29. The foregoing TCR of A28, wherein the α chain variable region comprises the amino acid sequence set forth in SEQ ID NO: 7, and the β chain variable region comprises the amino acid sequence set forth in SEQ ID NO: 8.
A30. The foregoing TCR of any one of A1-A29, wherein the TCR is recombinantly expressed, and/or expressed from a vector.
A31. The foregoing TCR of any one of A18-A30, wherein the α chain constant region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 9 or SEQ ID NO: 10.
A32. The foregoing TCR of A31, wherein the α chain constant region comprises the amino acid sequence set forth in SEQ ID NO: 9 or SEQ ID NO: 10.
A33. The foregoing TCR of any one of A18-A32, wherein the β chain constant region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 11, SEQ ID NO: 12, SEQ ID NO: 13, or SEQ ID NO: 14.
A34. The foregoing TCR of A33, wherein the β chain constant region comprises the amino acid sequence set forth in SEQ ID NO: 11, SEQ ID NO: 12, SEQ ID NO: 13, or SEQ ID NO: 14.
A35. The foregoing TCR of any one of A1-A34, wherein the TCR is recombinant.
A36. In certain non-limiting embodiments, the presently disclosed subject matter provides a T cell receptor (TCR) comprising an extracellular domain that binds to the same RAS peptide as a reference TCR or a functional fragment thereof, wherein the reference TCR or functional fragment thereof comprises an α chain variable region and a β chain variable region, wherein the α chain variable region comprises a CDR1 comprising the amino acid sequence set forth in SEQ ID NO: 16, a CDR2 comprising the amino acid sequence set forth in SEQ ID NO: 17, and a CDR3 comprising the amino acid sequence set forth in SEQ ID NO: 18; and the β chain variable region comprises a CDR1 comprising the amino acid sequence set forth in SEQ ID NO: 19, a CDR2 comprising the amino acid sequence set forth in SEQ ID NO: 20, and a CDR3 comprising the amino acid sequence set forth in SEQ ID NO: 21.
B1. In certain non-limiting embodiments, the presently disclosed subject matter provides a nucleic acid encoding the T cell receptor of any one of A1-A36.
C1. In certain non-limiting embodiments, the presently disclosed subject matter provides a cell comprising the TCR of any one of A1-A36 or the nucleic acid of B1.
C2. The foregoing cell of C1, wherein the cell is transduced with the TCR.
C3. The foregoing cell of C1 or C2, wherein the TCR is constitutively expressed on the surface of the cell.
C4. The foregoing cell of any one of C1-C3, wherein the cell is an immunoresponsive cell.
C5. The foregoing cell of any one of C1-C4, wherein the cell is selected from the group consisting of a T cell, a Natural Killer (NK) cell, and a pluripotent stem cell from which a lymphoid cell may be differentiated.
C6. The foregoing cell of C5, wherein the cell is a T cell.
C7. The foregoing cell of C6, wherein the T cell is selected from the group consisting of a cytotoxic T lymphocyte (CTL), a regulatory T cell, a γδ T cell, a Natural Killer-T cell (NK-T), a stem cell memory T cell, a central memory T cell, and an effector memory T cell.
C8. The foregoing cell of C7, wherein the T cell is a γδ T cell.
C9. The foregoing cell of C7, wherein the T cell is a NK-T cell.
C10. The foregoing cell of C5, wherein the cell is a NK cell
C11. The foregoing cell of any one of C1-C10, wherein the TCR or the nucleic acid is integrated at a locus within the genome of the cell.
C12. The foregoing cell of C11, wherein the locus is selected from the group consisting of a TRAC locus, a TRBC locus, a TRDC locus, and a TRGC locus.
C13. The foregoing cell of C11 or C12, wherein the locus is a TRAC locus or a TRBC locus.
C14. The foregoing cell of C11, wherein the locus is selected from a PDCD1 locus, a CBLB locus, a CISH locus, and a RASA2 locus.
C15. The foregoing cell of C14, wherein the locus is selected from a PDCD1 locus.
C16. The foregoing cell of C14, wherein the locus is selected from a CBLB locus.
C17. The foregoing cell of C14, wherein the locus is selected from a CISH locus.
C18. The foregoing cell of C14, wherein the locus is selected from a RASA2 locus.
C19. The foregoing cell of C11, wherein the locus is a genomic safe harbor.
C20. The foregoing cell of C1-C19, wherein the cell further comprises at least one recombinant or exogenous coreceptor.
C21. The foregoing cell of C20, wherein the co-receptor is a CD8 co-receptor.
C22. The foregoing cell of C20, wherein the co-receptor in a CD4 co-receptor.
D1. In certain non-limiting embodiments, the presently disclosed subject matter provides a composition comprising the cell of any one of C1-C22.
D2. The foregoing composition of D1, which is a pharmaceutical composition further comprising a pharmaceutically acceptable carrier.
E1. In certain non-limiting embodiments, the presently disclosed subject matter provides a vector comprising the nucleic acid of B1.
E2. The foregoing vector of E1, wherein the vector is a γ-retroviral vector.
F1. In certain non-limiting embodiments, the presently disclosed subject matter provides a method for producing a cell that binds to a RAS peptide that comprises a G12 mutation, comprising 25 introducing into the cell the nucleic acid of B1 or the vector of E1 or E2.
G1. In certain non-limiting embodiments, the presently disclosed subject matter provides a method of treating and/or preventing a tumor associated with RAS in a subject, comprising administering to the subject the cell of any one of C1-C22 or the composition of D1 or D2.
G2. The forgoing method of G1, wherein the tumor is associated with a RAS mutation.
G3. The forgoing method of G2, wherein the RAS mutation is a G12D mutation.
G4. The forgoing method of G2, wherein the RAS mutation is a G12V mutation.
G5. The forgoing method of G2, wherein the RAS mutation is a G12C mutation.
G6. The forgoing method of any one of G1-G5, wherein the tumor is selected from the group consisting of pancreatic cancer, breast cancer, endometrial cancer, cervical cancer, anal cancer, bladder cancer, colorectal cancer, cholangiocarcinoma/bile duct cancer, lung cancer, ovarian cancer, esophageal cancer, gastric cancer, head and neck squamous cell carcinoma, nonmelanoma skin cancer, salivary gland cancer, melanoma, and multiple myeloma.
G7. The forgoing method of G6, wherein the tumor is colorectal cancer.
G8. The forgoing method of any one of G1-G7, wherein the subject is a human G9. The forgoing method of any one of G1-G7, wherein the subject comprises an HLA-A.
G10. The forgoing method of G9, wherein the HLA-A is an HLA-A*03 superfamily member.
G11. The forgoing method of G10, wherein the HLA-A*03 superfamily member is selected from the group consisting of HLA-A*03, HLA-A*11, HLA-A*31, HLA-A*33, HLA-A*66, HLA-A*68 and HLA-A*74.
G12. The forgoing method of G10 or G11, wherein the HLA-A*03 superfamily member is HLA-A*11.
H1. In certain non-limiting embodiments, the presently disclosed subject matter provides a cell of any one of C1-C22 or the composition D1 or D2 for use in treating and/or preventing a tumor associated with RAS in a subject.
H2. The forgoing cell or composition for use of H1, wherein the tumor is associated with a RAS mutation.
H3. The forgoing cell or composition for use of H2, wherein the RAS mutation is a G12D mutation.
H4. The forgoing cell or composition for use of H2, wherein the RAS mutation is a G12V mutation.
H5. The forgoing cell or composition for use of H2, wherein the RAS mutation is a G12C mutation.
H6. The forgoing cell or composition for use of any one of H1-H5, wherein the tumor is selected from the group consisting of pancreatic cancer, breast cancer, endometrial cancer, cervical cancer, anal cancer, bladder cancer, colorectal cancer, cholangiocarcinoma/bile duct cancer, lung cancer, ovarian cancer, esophageal cancer, gastric cancer, head and neck squamous cell carcinoma, nonmelanoma skin cancer, salivary gland cancer, melanoma, and multiple myeloma.
H7. The forgoing cell or composition for use of H6, wherein the tumor is colorectal cancer.
H8. The forgoing cell or composition for use of any one of H1-H7, wherein the subject is a human.
H9. The forgoing cell or composition for use of any one of H1-H8, wherein the subject is a comprises an HLA-A.
H10. The forgoing cell or composition for use of H9, wherein the HLA-A is an HLA-A*03 superfamily member.
H11. The forgoing cell or composition for use of H10, wherein the HLA-A*03 superfamily member is selected from the group consisting of HLA-A*03, HLA-A*11, HLA-A*31, HLA-A*33, HLA-A*66, HLA-A*68 and HLA-A*74.
H12. The forgoing cell or composition for use of H10 or H11, wherein the HLA-A*03 superfamily member is HLA-A*11.

### EXAMPLES

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the compositions, and assay, screening, and therapeutic methods of the invention, and are not intended to limit the scope of what the inventors regard as their invention.

### Example 1

RAS proteins, comprising KRAS, HRAS and NRAS are encoded by the most commonly mutated oncogene family in cancer (27%). Mutations in KRAS represent 84% of RAS family mutations. As depicted in Table 2 below, RAS mutations are frequently encountered in colorectal, pancreatic and lung cancer, NRAS mutations in AML and cutaneous melanoma, and HRAS in bladder and head and neck cancers. The majority of non-synonymous mutations (91%) in KRAS occur at the p.G12 position and approximately 40% of these mutations are represented by a G>D substitution (FIGS. 1A and 1C). As shown in FIG. 1B, all four members of the RAS family share 90% sequence homology throughout their G domains but differ significantly in their N-terminal membrane targeting domains. Of note, the amino acid sequences surrounding the codon 12 hotspot region share 100% sequence homology between RAS family members. This suggests that a TCR specific for KRAS(G12D) might afford cross-protection to other mutant RAS proteins.

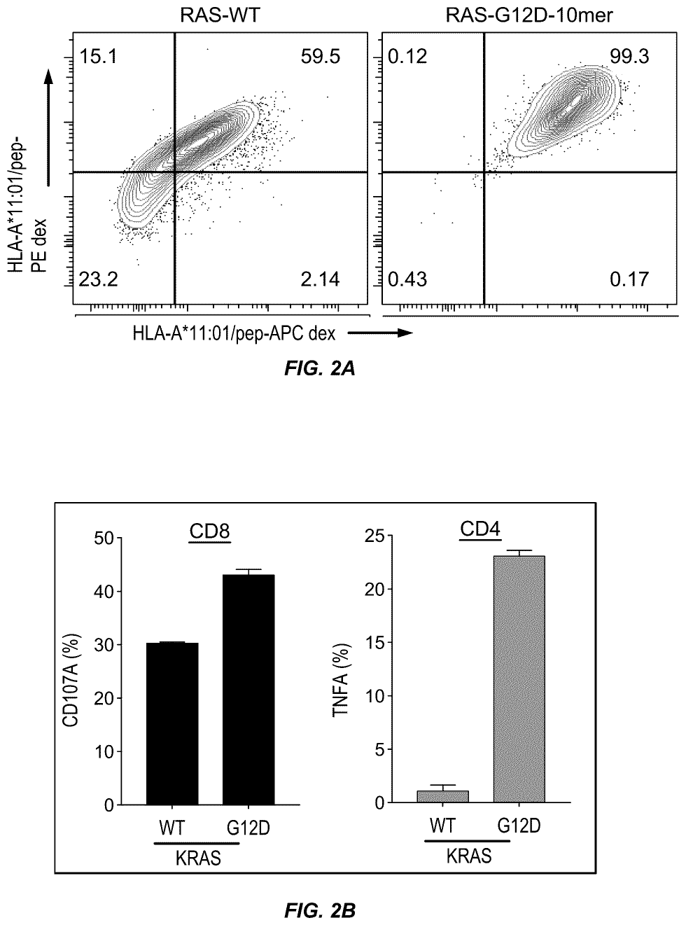

Here, the presently disclosed subject matter describes a novel T cell receptor (TCR T3) isolated from a colorectal cancer patient that recognizes mutated G12D KRAS in the context of HLA-A*11. To study the properties of this TCR, dual dextramer labeling of T cells (FIG. 2A). Open repertoire (non-specific) T cells were retrovirally transduced with a transgene encoding TCR T3. T cells were labeled with dextramers composed of HLA-A*11:01 complexes loaded with either the RAS-WT or RAS-G12D peptide. A fraction (approx. 60%) of the T cells bound the WT pMHC dextramers but preferentially bound the mutant peptide loaded complexes. Next, it was evaluated the specificity of TCR T3 transduced T cells (FIG. 2B). Function of TCR T3 transduced T cells was measured by coculturing with HLA-A*11:01⁺ target cells co-transfected with mRNA encoding either full-length wild type (WT) KRAS or KRAS(G12D). Upregulation of CD107A was determined in CD8⁺ (left, black) and TNF-α in CD4⁺ (right, red) T cells expressing the transduced TCR. Overall, these data demonstrated that CD8+ TCR T cells preferentially recognized mutant KRAS compared to WT KRAS while CD4⁺ T cells selectively recognized only mutant KRAS.

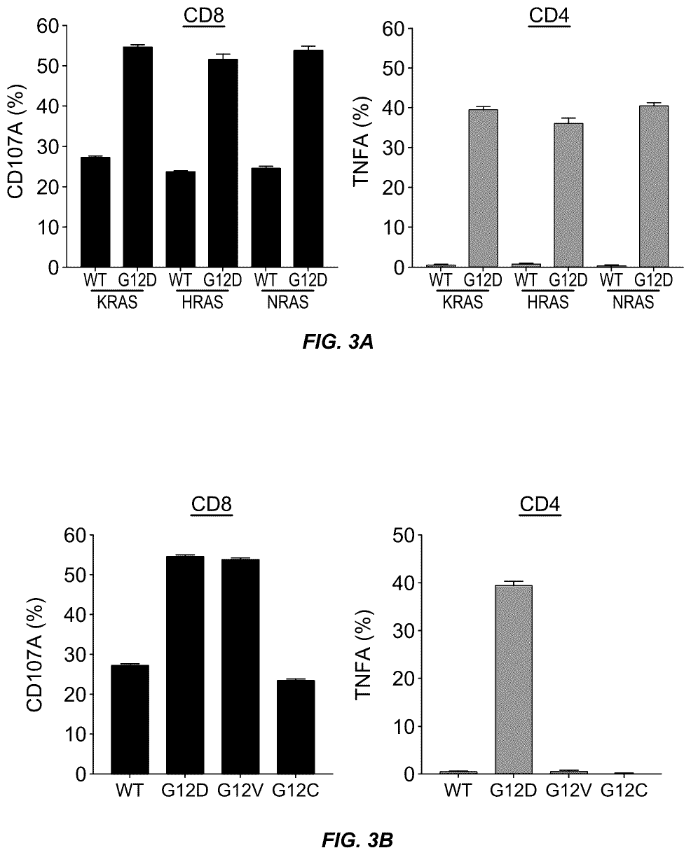

Next, the presently disclosed subject matter studied the cross-protection of RAS public neoantigen-specific TCR T3 against alternative mutant RAS variants. Open repertoire (non-specific) T cells were transduced with the TCR T3 gene sequence and co-cultured with HLA-A*11:01⁺ targets co-expressing individual RAS isoforms (KRAS, HRAS and NRAS). Corresponding WT RAS isoforms were used as specificity controls. As shown in FIG. 3A, upregulation of CD107A was determined in CD8⁺ (left, black) and TNF-α in CD4⁺ (right, red) T cells expressing the transduced TCR. CD8⁺ TCR T cells preferentially recognized mutant RAS isoforms while CD4⁺ T cells selectively recognized only the mutant RAS isoforms. Next, open repertoire (non-specific) T cells were transduced with the TCR T3 gene sequence and co-cultured with HLA-A*11:01⁺ targets co-expressing individual RAS hot spot mutations (G12V and G12C). G12D KRAS was included to establish degree of cognate epitope recognition. Upregulation of CD107A was determined in CD8+(left, black) and TNF-a in CD4⁺ (right, red) T cells expressing the transduced TCR (FIG. 3B). CD8⁺ TCR T cells recognized alternative RAS G12V and G12C substitutions, while CD4⁺ TCR T cells selectively recognized only RAS G12D.

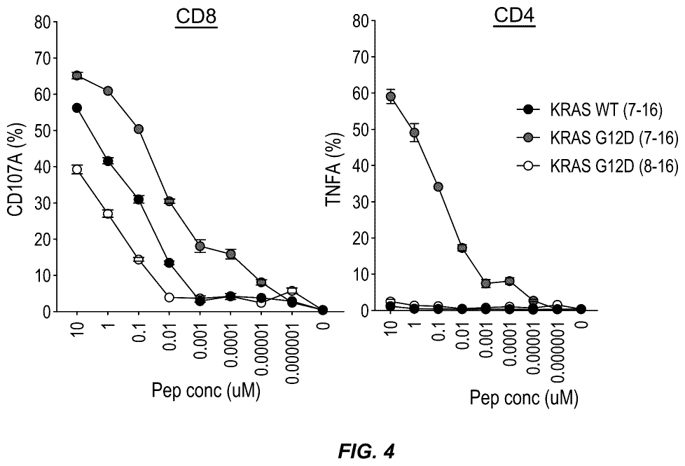

Finally, peptide-titration of T cells transduced with TCR T3 was performed. Open-repertoire T cells were retrovirally transduced with the TCR T3 gene sequence and cocultured with HLA-A*11:01⁺ targets pulsed with titrating amounts of either RAS(G12D) 7-16 (10-mer) or 8-16 (9-mer) neopeptides. WT 7-16 (10-mer) peptide was included as a control. Upregulation of CD107A was determined in CD8⁺ (left) and TNF-α in CD4⁺ (right) T cells expressing the transduced TCR. As demonstrated in FIG. 4, CD8⁺ T cells recognized titrating amounts of both WT and Mutant G12D peptides, while CD4 T cells selectively recognized only mutant peptides.

### EMBODIMENTS OF THE PRESENTLY DISCLOSED SUBJECT MATTER

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

All patents and publications and sequences referred to by accession or reference number mentioned in this specification are herein incorporated by reference to the same extent as if each independent patent and publication and sequence was specifically and individually indicated to be incorporated by reference.

What is claimed is:

1. An isolated T cell receptor (TCR) comprising an extracellular domain that binds to a RAS peptide comprising a G12 mutation,
wherein the extracellular domain comprises (a) an α chain comprising a variable region and a constant region, said variable region comprising a CDR1 comprising the amino acid sequence set forth in SEQ ID NO: 16, a CDR2 comprising the amino acid sequence set forth in SEQ ID NO: 17, and a CDR3 comprising the amino acid sequence set forth in SEQ ID NO: 18; and (b) a β chain comprising a variable region and a constant region, said variable region comprising a CDR1 comprising the amino acid sequence set forth in SEQ ID NO: 19, a CDR2 comprising the amino acid sequence set forth in SEQ ID NO: 20, and a CDR3 comprising the amino acid sequence set forth in SEQ ID NO: 21.

2. The TCR of claim 1, wherein the RAS peptide comprises a G12D mutation, a G12V mutation, or a G12C mutation.

3. The TCR of claim 2, wherein the RAS peptide comprises or consists of the amino acid sequence set forth in SEQ ID NO: 1, SEQ ID NO: 2, SEQ ID NO: 4, SEQ ID NO: 5, or SEQ ID NO: 6.

4. The TCR of claim 1, wherein the α chain variable region comprises an amino acid sequence that is at least about 80% identical to the amino acid sequence set forth in SEQ ID NO: 7, and/or the β chain variable region comprises an amino acid sequence that is at least about 80% identical to the amino acid sequence set forth in SEQ ID NO: 8.

5. The TCR of claim 1, wherein the α chain variable region comprises the amino acid sequence set forth in SEQ ID NO: 7, and/or the β chain variable region comprises the amino acid sequence set forth in SEQ ID NO: 8.

6. The TCR of claim 1, wherein the α chain constant region comprises an amino acid sequence that is about 80% identical to the amino acid sequence set forth in SEQ ID NO: 9, SEQ ID NO: 10, or SEQ ID NO: 25; and/or the β chain constant region comprises an amino acid sequence that is about 80% identical to the amino acid sequence set forth in SEQ ID NO: 11, SEQ ID NO: 12, SEQ ID NO: 13, or SEQ ID NO: 14.

7. The TCR of claim 1, wherein the α chain constant region comprises the amino acid sequence set forth in SEQ ID NO: 9, SEQ ID NO: 10, or SEQ ID NO: 25; and/or the β chain constant region comprises the amino acid sequence set forth in SEQ ID NO: 11, SEQ ID NO: 12, SEQ ID NO: 13, or SEQ ID NO: 14.

8. A nucleic acid encoding the T cell receptor (TCR) of claim 1.

9. A cell comprising an exogenous T cell receptor (TCR) comprising an extracellular domain that binds to a RAS peptide comprising a G12 mutation,
wherein the extracellular domain comprises (a) an α chain comprising a variable region and a constant region, said variable region comprising a CDR1 comprising the amino acid sequence set forth in SEQ ID NO: 16, a CDR2 comprising the amino acid sequence set forth in SEQ ID NO: 17, and a CDR3 comprising the amino acid sequence set forth in SEQ ID NO: 18; and (b) a β chain comprising a variable region and a constant region, said variable region comprising a CDR1 comprising the amino acid sequence set forth in SEQ ID NO: 19, a CDR2 comprising the amino acid sequence set forth in SEQ ID NO: 20, and a CDR3 comprising the amino acid sequence set forth in SEQ ID NO: 21.

10. The cell of claim 9, wherein the cell is an immunoresponsive cell.

11. The cell of claim 10, wherein the cell is selected from the group consisting of a T cell, a Natural Killer (NK) cell, and a pluripotent stem cell from which a lymphoid cell may be differentiated.

12. The cell of claim 11, wherein the cell is a T cell.

13. The cell of claim 12, wherein the T cell is selected from the group consisting of a cytotoxic T lymphocyte (CTL), a regulatory T cell, a γδ T cell, a Natural Killer-T cell (NK-T), a stem cell memory T cell, a central memory T cell, and an effector memory T cell.

14. The cell of claim 13, wherein the T cell is a γδ T cell.

15. The cell of claim 13, wherein the T cell is an NK-T cell.

16. The cell of claim 11, wherein the cell is an NK cell.

17. The cell of claim 9, wherein the TCR is encoded by a nucleic acid integrated at a locus within the genome of the cell.

18. The cell of claim 17, wherein the locus is selected from a TRAC locus, a TRBC locus, a TRDC locus, a TRGC locus, a PDCD1 locus, a CBLB locus, a CISH locus, a RASA2 locus, or a genomic safe harbor.

19. The cell of claim 9, wherein the cell further comprises at least one recombinant or exogenous coreceptor.

20. The cell of claim 19, wherein the co-receptor is a CD8 co-receptor or a CD4 co-receptor.

21. A composition comprising the cell of claim 9, which is a pharmaceutical composition further comprising a pharmaceutically acceptable carrier.

22. A vector comprising the nucleic acid of claim 8.

23. The vector of claim 22, wherein the vector is a γ-retroviral vector.

24. A method for producing a cell that binds to a RAS peptide that comprises a G12 mutation, comprising introducing into the cell the nucleic acid of claim 21.

25. A method of treating and/or preventing a tumor associated with RAS in a subject, comprising administering to the subject the cell of claim 9 or a pharmaceutical composition thereof.

26. The method of claim 25, wherein the tumor is associated with a RAS mutation selected from a G12D mutation, a G12V mutation, or a G12C mutation.

27. The method of claim 25, wherein the tumor is selected from the group consisting of pancreatic cancer, breast cancer, endometrial cancer, cervical cancer, anal cancer, bladder cancer, colorectal cancer, cholangiocarcinoma/bile duct cancer, lung cancer, ovarian cancer, esophageal cancer, gastric cancer, head and neck squamous cell carcinoma, nonmelanoma skin cancer, salivary gland cancer, melanoma, and multiple myeloma.

28. The method of claim 25, wherein the subject comprises an HLA-A*03 superfamily member selected from the group consisting of HLA-A*03, HLA-A*11, HLA-A*31, HLA-A*33, HLA-A*66, HLA-A*68 and HLA-A*74.


SEQUENCE LISTING (25 sequences)

SEQ ID NO: 1 - protein, 9 aa, synthetic construct
  REGION 1..9: note = Description of sequence: RAS peptide componet
VVGADGVGK

SEQ ID NO: 2 - protein, 10 aa, synthetic construct
  REGION 1..10: note = Description of sequence: RAS peptide componet
VVVGADGVGK

SEQ ID NO: 3 - protein, 10 aa, synthetic construct
  REGION 1..10: note = Description of sequence: RAS peptide componet
VVVGAGGVGK

SEQ ID NO: 4 - protein, 9 aa, synthetic construct
  REGION 1..9: note = Description of sequence: RAS peptide componet
VVGAVGVGK

SEQ ID NO: 5 - protein, 10 aa, synthetic construct
  REGION 1..10: note = Description of sequence: RAS peptide componet
VVVGAVGVGK

SEQ ID NO: 6 - protein, 10 aa, synthetic construct
  REGION 1..10: note = Description of sequence: RAS peptide componet
VVVGACGVGK

SEQ ID NO: 7 - protein, 129 aa, synthetic construct
  REGION 1..129: note = Description of sequence: Alpha chain variable region
MLLEHLLIILWMQLTWVSGQQLNQSPQSMFIQEGEDVSMNCTSSSIFNTWLWYKQDPGEGPVLLIALYKAGELTSNGRLTAQFGITRKDSFLNISASIPSDVGIYFCAGRREGAQKLVFGQGTRLTINP

SEQ ID NO: 8 - protein, 134 aa, synthetic construct
  REGION 1..134: note = Description of sequence: beta chain variable region
MGTRLLCWVVLGFLGTDHTGAGVSQSPRYKVAKRGQDVALRCDPISGHVSLFWYQQALGQGPEFLTYFQNEAQLDKSGLPSDRFFAERPEGSVSTLKIQRTQQEDSAVYLCASSSTLMGVNIQYFGAGTRLSVL

SEQ ID NO: 9 - protein, 141 aa, Homo sapiens
  REGION 1..141: note = Description of sequence: Human ? chain constant region
NIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 10 - protein, 137 aa, Mus musculus
  REGION 1..137: note = Description of sequence: Mouse ? chain constant region
NIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 11 - protein, 177 aa, Homo sapiens
  REGION 1..177: note = Description of sequence: Human ? chain constant region
EDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 12 - protein, 179 aa, Homo sapiens
  REGION 1..179: note = Description of sequence: Human ? chain constant region
EDLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 13 - protein, 179 aa, Homo sapiens
  REGION 1..179: note = Description of sequence: Human ? chain constant region
EDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 14 - protein, 173 aa, Mus musculus
  REGION 1..173: note = Description of sequence: Mouse ? chain constant region
EDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 15 - protein, 27 aa, synthetic construct
  REGION 1..27: note = Description of sequence: furin-2A-peptide
RAKRSGSGATNFSLLKQAGDVEENPGP

SEQ ID NO: 16 - protein, 5 aa, synthetic construct
  REGION 1..5: note = Description of sequence: CDR1
SIFNT

SEQ ID NO: 17 - protein, 7 aa, synthetic construct
  REGION 1..7: note = Description of sequence: CDR2
LYKAGEL

SEQ ID NO: 18 - protein, 13 aa, synthetic construct
  REGION 1..13: note = Description of sequence: CDR3
CAGRREGAQKLVF

SEQ ID NO: 19 - protein, 5 aa, synthetic construct
  REGION 1..5: note = Description of sequence: CDR1
SGHVS

SEQ ID NO: 20 - protein, 6 aa, synthetic construct
  REGION 1..6: note = Description of sequence: CDR2
FQNEAQ

SEQ ID NO: 21 - protein, 15 aa, synthetic construct
  REGION 1..15: note = Description of sequence: CDR3
CASSSTLMGVNIQYF

SEQ ID NO: 22 - protein, 235 aa, synthetic construct
  REGION 1..235: note = Description of sequence: beta chain
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 23 - protein, 210 aa, synthetic construct
  REGION 1..210: note = Description of sequence: beta chain
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQFYK

SEQ ID NO: 24 - protein, 458 aa, synthetic construct
  REGION 1..458: note = Description of sequence: CD4 co-receptor
MNRGVPFRHLLLVLQLALLPAATQGKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 25 - protein, 134 aa, Mus musculus
  REGION 1..134: note = Description of sequence: Mouse ? chain constant region
DIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRL